(19) United States
(12) Patent Application Publication
(10) Pub. No.: US 2023/0062684 A1
(43) Pub. Date: Mar. 2, 2023

(54) INTRAVASCULAR THROMBECTOMY DEVICE AND PROCESS FOR TREATING ACUTE ISCHEMIC STROKE

(71) Applicants: Sheri Albers, Sacramento, CA (US); John Kucharczyk, Reno, NV (US); Gregory Latchaw, Wellington, FL (US); Johannes van Vaals, Lakewood Ranch, FL (US); Mark A. Litman, Edina, MN (US)

(72) Inventors: Sheri Albers, Sacramento, CA (US); John Kucharczyk, Reno, NV (US); Gregory Latchaw, Wellington, FL (US); Johannes van Vaals, Lakewood Ranch, FL (US); Mark A. Litman, Edina, MN (US)

(21) Appl. No.: 17/893,290

(22) Filed: Aug. 23, 2022

Related U.S. Application Data
(63) Continuation-in-part of application No. 17/460,210, filed on Aug. 28, 2021.

(51) Int. Cl. A61B 17/221

(52) U.S. Cl. CPC A61B 17/221; A61B 2017/00867

(57) ABSTRACT

An intravascular thrombus retraction device includes: wires that are compressible into a compact cylindrical form within a catheter and are self-expandable into a wire mesh web with at least some parallel wires forming openings in the wire mesh sufficient to allow fluid passage and small enough to filter particles of at least 0.001 mm, a base of the wire mesh web connected to radial ring-shaped structure supporting and maintaining an opening in the base of the wire mesh and forming a thrombus capture volume, and the radial ring-shaped structure being compressible into the catheter and being self-expandable or expandable by struts when free of compressive forces within the catheter to open up into an open, expanded, radial ring-shaped structure which maintains the opening in the opening in the base of the wire mesh.

### RELATED APPLICATIONS DATA

This application claims priority under 35 U.S.C. 120 as a continuation-in-part application from US Pat. Application Serial No 17/460,210, filed 28 Aug. 2021.

### BACKGROUND OF THE INVENTION

### 1. Field of the Invention

This invention pertains to intravascular medical devices for isolating, capturing, and removing blood clots from a blood vessel. This same system may also be used to retrieve obstructions, using coils, balloons, or catheter fragments dislodged during interventional procedures from the blood stream. The same system may also be used to remove obstructions from ducts and other cavities of the body, for example, foreign bodies or stones from the urinary or the biliary tracts. In particular, this invention relates to medical devices for the intravascular treatment of acute ischemic stroke (AIS), deep vein thrombosis (DVT) and acute pulmonary embolism (PE).

### 2. Background of the Art

In acute ischemic stroke (AIS), the density of occluding clots is related to the content of fibrin content of red blood cells. However, no CT or MRI marker for fibrin content has been established. The present technology uses the mechanical thrombectomy device of the present invention te-for isolating, capturing, and removing blood clots from a blood vessel. Blood clots are made up of platelets and a meshwork of protein fibrin strands. Clots in arteries have a different composition than clots in veins. Two main types of blood clots include the thrombus or the embolus. Clots in arteries are mostly made up of platelets. Clots in veins are mostly made up of fibrin. Studies have been made of captured clot materials, and the present technology can advance those studies by retrieving more intact thrombi.

“Clot composition and treatment approach to acute ischemic stroke: The road so far, Annals of Indian Academy of Neurology, 2013 Oct-Dec; 16(4): 494-497, Paramdeep Singh, Rupinderjeet Kaur, and Amarpreet Kaur shows that recent histological studies of thrombi retrieved from patients with an acute ischemic stroke using the endovascular thrombectomy devices and correlation with early vessel computed tomography (CT) and magnetic resonance imaging (MRI) characteristics have given relevant insights into the pathophysiology of thrombotic lesions and may facilitate the development of improved reperfusion treatment approaches. We present a review of recent studies on the histopathologic analysis of thrombi, studies of MRI, and CT imaging correlation with thrombus histology, and detailed structural analysis of thrombo-emboli retrieved by thrombectomy devices during an AIS acute ischemic stroke.

A first histopathological evaluation of cerebral thrombi was carried out by Torvik and Jorgensen (Torvik A, Joergensen L. Thrombotic Embolic occlusions of the carotid arteries in an autopsy material. i. prevalence, location and associated diseases. J Neurol Sci. 1964;41:24-39) more than 50 years ago in post-mortem cases with one and half month old obstruction. Recently, mechanical thrombectomy devices have provided an opportunity to directly investigate fresh thrombi specimens by retrieving them from the target arteries in patients with an acute ischemic stroke. On the whole, it is not known whether the choice of therapies ought to take into consideration for composition of thrombus. A crucial issue is whether the structure of a thrombus ascertains its appearance on magnetic resonance imaging (MRI) and computed tomography (CT) and whether the thrombus structure also determines its susceptibility to thrombolytic agents like tissue plasminogen activator (tPA).

Examination of freshly retrieved thrombi from patients with an acute ischemic stroke could help to improve knowledge of stroke pathophysiology, and soon might be expected to play a role in shaping success of treatment approaches after patient selection. A review of human and animal studies was carried out on the histopathologic evaluation of thrombi, studies of MRI and CT imaging correlation with thrombus histology, and detailed structural analysis of thromboemboli by scanning electron microscopy (SEM).

Marder et al. (Marder VJ, Chute DJ, Starkman S, Abolian AM, Kidwell C, Liebeskind D, et al., “Analysis of thrombi retrieved from cerebral arteries of patients with acute ischemic stroke.” Journal Stroke. 2006;37:2086-93; Published online Sep 30. 2021 doi: 10.5853/jos.2021.02306.)did the systematic systematic histological analysis of thromboemboli retrieved by Merci™ retriever from the middle cerebral artery and intracranial carotid artery of 25 patients with acute ischemic stroke within or beyond 8 h of symptom onset. Despite the presence of common components of the fibrin-platelets, nucleated cells (neutrophil/monocyte) and red blood cells (RBC’s) in all cases, there was a large diversity of histological pattern as well as in quantitative proportion of different components. The authors accredited this to haphazard and disorganized forces of sheer and turbulence imposed on site of thrombus formation contradicting long established system of belief that a cardiac source with slow flow causes the formation of “red” (erythrocyte) clots, whereas high flow in arteries formed “white” (fibrin) clots. Since there was heterogeneity of clot composition, traditional definition of red versus white clots was not found to be truly applicable.

A separate study in “Clot Composition Analysis as a Diagnostic Tool to Gain Insight into Ischemic Stroke Etiology: A Systematic Review,” Alicia Aliena-Valero, Julia Baixauli-Martín, Germán Torregrosa, Jose I. Tembl, and Juan B. Salom stated that: Mechanical thrombectomy renders the occluding clot available for analysis. Insights into thrombus composition could help establish the stroke cause. We investigated the value of clot composition analysis as a complementary diagnostic tool in determining the etiology of large vessel occlusion (LVO) ischemic strokes (International Prospective Register of Systematic Reviews (PROSPERO) registration # CRD42020199436). Following the Preferred Reporting Items for Systematic Reviews and Meta-Analyses (PRISMA) guidelines, we ran searches on Medline (using the PubMed interface) and Web of Science for studies reporting analyses of thrombi retrieved from LVO stroke patients subjected to mechanical thrombectomy (Jan. 1, 2006 to Sep. 21, 2020). The PubMed search was updated weekly up to Feb. 22, 2021. Reference lists of included studies and relevant reviews were hand-searched. From 1,714 identified studies, 134 eligible studies (97 cohort studies, 31 case reports, and six case series) were included in the qualitative synthesis. Physical, histopathological, biological, and microbiological analyses provided information about the gross appearance, mechanical properties, structure, and composition of the thrombi. There were non-unanimous associations of thrombus size, structure, and composition (mainly proportions of fibrin and blood formed elements) with the Trial of Org 10172 in Acute Stroke Treatment (TOAST) etiology and underlying pathologies, and similarities between cryptogenic thrombi and those of known TOAST etiology. Individual thrombus analysis contributed to the diagnosis, mainly in atypical cases. Although cohort studies report an abundance of quantitative rates of main thrombus components, a definite clot signature for accurate diagnosis of stroke etiology is still lacking. Nevertheless, the qualitative examination of the embolus remains an invaluable tool for diagnosing individual cases, particularly regarding atypical stroke causes.

Ovine blood clot types containing defined amounts of red blood cells, mixed clots and red clots may be scanned in a SDCT evaluation test (e.g., IQon®, Philips) (a) in a tube containing saline, (b) 5 min and (c) 3 days after exposure to a 1:50 dilution of contrast iohexol (e.g., Accupaque-350®). The attenuation of the clots can be measured in nonenhanced and contrast-enhanced SDCT. Statistical analysis can be conducted with ANOVA and linear and multivariable regression models. Dual-energy CT provided by the IQon® CT system is often used in routine examinations-from thoracic to brain, to neck and spine. Approximately 50% of these exams are performed with injections of iodinated contrast to highlight blood vessels and enhance organ or lesion structures. However, in some patients, the amount of contrast media delivered is of concern due to risk of complications, such as contrast-induced nephropathy (CIN). Additionally, the ability of Monoenergetic (MonoE) spectral results from a multi-detector CT to boost iodine signal at low keV’s allows for improved visualization of structures at low volumes of iodinated contrast.

Powering the IQon’s unique capabilities is Philips’ proprietary NanoPanel Prism spectral detector technology. Utilizing a detector-based versus source-based system to capture spectral data, the NanoPanel Prism detector simultaneously distinguishes and captures high and low photon energy-improving visualization and characterization of tissues and structures while enhancing image quality.

Projection space spectral reconstruction provides a single DICOM entity, known as Spectral Base Image (SBI), that contains sufficient information for retrospective analysis. Spectral applications create various high quality spectral results from the SBI. There is no indication in any literature of SDCT being used to identify properties of thrombus, although detection and confirmation of Thrombus has been reported. (APPLIED RADIOLOGY, IQon® Spectral CT in the Diagnosis of Pulmonary Embolism and Left Ventricular Thrombus.

Previous studies have reported that fibrin content in blood clots is related to contrast uptake. It is to be determined whether after prolonged exposure, fibrin rich clots will show a significant increase of density due to further uptake of contrast medium. The density of the clot formations in native CT scans will be evaluated in terms of the RBC content. CT density and relative enhancement of clots are potentially independent determinants of clot composition.

Mechanical thrombectomy devices enable thrombi from target arteries in patients with AIS to will be investigated. Evaluation of retracted thrombi from patients with AIS could help elucidate stroke pathophysiology.

IMCAT proposes to conduct histological analyses of thrombus from the middle cerebral artery and intracranial carotid artery of patients in AIS patients. Differences between red versus white clots will also be evaluated, including thrombus histology, stroke etiology, site of occlusion and reperfusion status. Calcific deposits and cholesterol crystals will also be evaluated in the thrombus specimens.

The present invention pertains generally to thrombus that may produce a clot in a patient’s vasculature. Clots can restrict blood flow to body tissues, in which blockage or obstruction may lead to serious medical consequences, including AIS, DVT and PE. When a clot forms in the venous circulation, it may lodge within a pulmonary blood vessel causing PE. A PE can decrease blood flow through the lungs, which in turn causes decreased oxygenation of the lungs, heart, and rest of the body.

One step in the removal of a thrombus is to determine certain rheological properties of the clot. One property that is of particular importance is the viscoelastic properties of the thrombus. Viscoelastic properties can indicate how likely a blood clot is to break apart during removal, and thus must be accurately measured or estimated prior to attempted removal of a thrombus.

It is important to do all measurements of the rheological properties of the thrombus through noninvasive means. Invasive means may disturb the blood clot, resulting in the thrombus breaking apart and potentially causing embolisms. Noninvasive methods of determining the rheological properties of the blood clot avoids the risk of the thrombus breaking apart and the potential for embolisms.

As the viscoelastic properties of a thrombus vary greatly between the acute, sub-acute, and chronic Deep Vein Thrombosis (DVT) periods, the method of removal becomes dependent on the thrombus’s age. For acute thrombi, which are composed mostly of platelets, fibrin, and neutrophils, there is a higher risk of detachment and thus a higher risk of Pulmonary Embolisms (PE). On the other hand, for chronic thrombi, where the acute thrombi’s components are replaced by collagen and mononuclear cells, the blood clot hardens progressively over time. Furthermore, while acute DVTs are treated with oral anticoagulants along with heparin, chronic thrombi are traditionally treated with oral anticoagulants alone. Therefore, it is imperative to determine the viscoelastic properties of the DVT, along with the estimated age and maturity of the DVT, for determining the optimal treatment.

Conventional approaches to treating thromboembolism include clot reduction and/or removal. Anticoagulants can prevent additional clots from forming, and thrombolytics can be partially disintegrate the clot. However, such agents typically take a prolonged period of time and, in some instances, can induce hemorrhage. Transcatheter clot devices can cause trauma to the vessel, are hard to navigate to the pulmonary embolism site, and may be expensive to manufacture. Surgical procedures come with increased cost, procedure time, risk of infection, higher morbidity, higher mortality, and recovery time. Accordingly, there is need for better devices and methods.

DVT and PE are considered as part of the same venous thromboembolism (VTE) disease process. The most frequent long-term complication of DVT is post-thrombotic syndrome (PTS). Veins in the leg or pelvis are most commonly affected, including the popliteal vein, femoral vein, iliac veins of the pelvis, and the inferior vena cava. Upper extremity DVT most commonly affects the subclavian, axillary, and jugular vein.

Acute PE represents the most serious clinical manifestation of VTE disease. In patients with hemodynamically significant PE, systemic thrombolysis improves right ventricular dysfunction and reduces pulmonary artery pressures. However, systemic thrombolysis is associated with a risk of bleeding, particularly intracranial hemorrhage. An alternative to direct infusion into the pulmonary artery using an infusion catheter may provide the benefit of clot retraction to reduce the risk of bleeding.

Venous duplex ultrasonography is the gold-standard for imaging DVT, but this method is insufficient for determining a thrombi’s age. Therefore, ultrasonic elastography uses ultrasonic frequencies on the thrombi’s deformation to assess the local hardness and therefore the thrombi’s age. Quasi-static ultrasound elastography is the most traditionally used method for ultrasonic elastography, and this method uses radiofrequency echo signals before and after a small, applied deformation and correlates it with tissue displacements. The tissue displacements can then be used to measure the tissue’s strain tensors or tissue’s Young’s modulus, which then can be correlated with the thrombi’s hardness and age. However, with most ultrasonic measuring systems, the inconsistent distribution of components within thrombi and the tissue surrounding the thrombi (such as blood vessels) when measuring non-invasively can create errors when determining the viscoelasticity of the thrombus.

One method for determining the viscoelasticity of biological tissues is to use Shear-Wave Dispersion Ultrasound Vibrometry (SDUV), and this method has been shown to work with thrombus. In this method, tissue vibration is generated through a focused ultrasound beam, resulting in shear waves propagating outwards from the focal zone of the beam. This wave propagation leads to tissue displacement, and the resulting phase change of shear wave is measured by another ultrasound transducer at a certain distance. This is used to calculate the speed of shear wave propagation in the tissue. The shear wave propagation speed through tissue is calculated at multiple frequencies, and this is then used to estimate the viscoelastic properties of the thrombus. SDUV can be performed with commercially available ultrasound equipment, such as the Aplio i-series by Canon Medical Systems.

The grasping element compatible with the removal of the clot should be used by the practitioner to remove the thrombus. Compatible means capable of properly removing the clot given its measured rheological properties such that the clot shall not break apart in a way that parts of the clot are not captured.

Among the U.S. patents that deal with relevant ultrasound techniques are:

U.S. Pat. No. 10,448,924 (Fraser) describes an ultrasonic diagnostic imaging system for shear wave measurement transmits push pulses in the form of a sheet of energy. The sheet of energy produces a shear wavefront which is a plane wave, which does not suffer from the 1/R radial dissipation of push pulse force as does a conventional push pulse generated along a single push pulse vector. The sheet of energy can be planar, curved, or in some other two or three dimensional shape. A curved sheet of energy can produce a shear wave source which focuses into a thin line, which increases the resolution and sensitivity of the measuring techniques used to detect the shear wave effect.

U.S. Pat. No. 10,675,000 (Specht) describes that changes in tissue stiffness have long been associated with disease. Systems and methods for determining the stiffness of tissues using ultrasonography may include a device for inducing a propagating shear wave in tissue and tracking the speed of propagation, which is directly related to tissue stiffness and density. The speed of a propagating shear wave may be detected by imaging a tissue at a high frame rate and detecting the propagating wave as a perturbance in successive image frames relative to a baseline image of the tissue in an undisturbed state. In some embodiments, sufficiently high frame rates may be achieved by using a ping-based ultrasound imaging technique in which unfocused omni-directional pings are transmitted (in an imaging plane or in a hemisphere) into a region of interest. Receiving echoes of the omnidirectional pings with multiple receive apertures allows for substantially improved lateral resolution.

U.S. Pat. No. 9,345,448 (Fatemi) describes a system and method for determining viscoelasticity of curved tissue walls using ultrasound bladder vibrometry (UBV). The UBV is a non-invasive technique utilizing, in a specific case, a focused ultrasound radiation force to excite Lamb waves in a curved bladder wall and pulse-echo techniques to track the tissue deformation propagating through such curved wall. Cross-spectral analysis is used to calculate the wave velocity, which is directly related to the elastic properties of the bladder wall.

U.S. Pat. No. 10,365,254 (Chen) describes assessment of blood coagulation using an acoustic radiation force based optical coherence elastography (ARF-OCE). An apparatus and method of using an optical coherence elastography (OCE) under acoustic radiation force (ARF) excitation includes the steps of inducing an excitation wave in a blood sample by use of an ultrasound beam from an ultrasonic transducer; measuring an elastic property of the blood sample by use of an optical coherence tomography (OCT) beam transverse to the ultrasound beam to dynamically measure the elastic property of the blood sample during coagulation and assessing the clot formation/dissolution kinetics and strength.

U.S. Pat. No. 10,926,112 (Powers) describes ultrasonic sonothrombolysis systems to produce two acoustic pressure levels of insonation during stroke therapy, mid/high acoustic pressure insonation directed to the site of a blood clot where microbubbles are present to induce microbubble-mediated blood clot lysis, and low acoustic insonation directed to the region surrounding the site of the blood clot where microbubbles are present to stimulate microvascular reperfusion of the surrounding tissue. The systems simultaneously produce blood clot lysis at the site of an occlusion and stimulate reperfusion of tissue affected by the occlusion.

U.S. Pat. Nos. 10,495,613 and 11,002,712 (Walker) describes a device for estimating a mechanical property of a sample is disclosed herein. The device may include a chamber configured to hold the sample; a transmitter configured to transmit a plurality of waveforms, including at least one forcing waveform; and a transducer assembly operatively connected to the transmitter and configured to transform the transmit waveforms into ultrasound waveforms. The transducer assembly can also transmit and receive ultrasound waveforms into and out of the chamber, as well as transform at least two received ultrasound waveforms into received electrical waveforms. The device also includes a data processor that can receive the received electrical waveforms; estimate a difference in the received electrical waveforms that results at least partially from movement of the sample; and estimate a mechanical property of the sample by comparing at least one feature of the estimated difference to at least one predicted feature, wherein the at least one predicted feature is based on a model of an effect of the chamber wall. Finally, the device can also include a controller configured to control the timing of the ultrasound transmitter and data processor.

U.S. Pat. No. 10,962,524 (Viola) describes an integrated system for determining a hemostasis and oxygen transport parameter of a blood sample, such as blood, is disclosed. The system includes a measurement system, such as an ultrasonic sensor, configured to determine data characterizing the blood sample. For example, the data could be displacement of the blood sample in response to ultrasonic pulses. An integrated aspect of the system may be a common sensor, sample portion or data for fast and efficient determination of both parameters. The parameters can also be used to correct or improve measured parameters. For example, physiological adjustments may be applied to the hemostatic parameters using a HCT measurement. Also, physical adjustments may be applied, such as through calibration using a speed or attenuation of the sound pulse through or by the blood sample. These parameters may be displayed on a GUI to guide treatment.

Although the unique thrombus capture device of the present technology is designed to have broad-range utility for soft, medium and hard clots/thrombi, there are also disclosed optimized structures within the generic disclosure of the device that can be selected to enhance performance with specifically identified thrombus rheologies. For example, the mesh size on the baskets and expander may be modified because harder clots are likely to generate smaller and more numerous emboli during the procedure, so a finer mesh opening size may be desirable when a hard clot has been identified. On the other hand, a finer mesh size may act to shred a softer clot, so that a larger mesh size might be desirable.

Once AIS, DVT or PE has been diagnosed, treatments can range from anticoagulation alone, catheter-directed thrombolysis, full-dose systemic thrombolysis, reduced-dose systemic thrombolysis, catheter embolectomy, or surgical embolectomy.

There is a review of the analysis of clot properties regarding stroke morphology which can assist in the selection of treatments and prescription of treatments to avoid similar strokes published as

Thrombus Composition, Imaging, and Outcome Prediction in Acute Ischemic Stroke; Raed A. Joundi, Bijoy K. Menon; First published Nov. 16, 2021, DOI: https://doi.org10.1212/WNL.0000000000012796; Neuroimaging. This article/review concludes and evidences that:

Advancing understanding of intracranial thrombus will require parallel advancement in both in vitro and in vivo studies. The Clot Summit Group highlighted the high heterogeneity between in vitro thrombus composition studies and outlined several ways to improve study of intracranial thrombi for consistency and reliability. These measures include improving quantitative analysis of thrombus composition, studying the whole thrombus specimen, standardizing methods, and building a central repository for retrieved clots. Thrombus registries are underway to better understanding thrombus composition in the modern EVT era (BACTRAC, STRIP, COMPO-CLOT). Together with recent published best practice guidelines for thrombus retrieval, processing, and analysis, further insights may soon be gained on thrombus composition in stroke. Advanced imaging methods that more directly assess the thrombus in vivo are emerging, such as use of nanoparticles, automated thrombus segmentation, electron microscopy, or radiomics. Proteomics may also be an important new method to detect and characterize novel biomarkers related to the pathophysiology of thrombus formation in stroke, particularly in cryptogenic stroke, and complement histologic analyses. Furthermore, machine learning and artificial intelligence are promising approaches that may improve the accuracy, reliability, and efficiency of clot characterization. Ultimately, the most effective and revealing studies will simultaneously evaluate detailed radiologic features, in vitro thrombus analysis, radiologic outcomes (i.e., recanalization and infarct volume), and clinical outcomes (i.e., disability).

The advent of rapid care pathways for acute stroke towards thrombolysis and EVT has modified our understanding of thrombus characteristics and treatment selection. As treatment moves away from strictly time-based to imaging-based patient selection and therapeutics, we need to improve our ability to prognosticate, select appropriate patients for treatment, and employ the correct treatment for each patient in the most rapid fashion possible. Despite many advances in our knowledge of thrombus characteristics and imaging, clear recommendations have yet to emerge on how to apply this knowledge in the individualized selection of reperfusion therapy. Improving decision-making will require continued parallel progress throughout the entire spectrum of stroke research, including thrombus characteristics, effective use of imaging modalities, and expansion of interventional tools and technology.

Various medical devices have been used commercially in treating DVT and PE, including examples disclosed by U.S. Pat. Nos. 10,238,406, 10,524,811, 10,342,571, 10,098,651, 10,045,790, 10,588,655, 10,349,690, 10,335,186, 10,231,751, 9,844,387, 9,700,332, 9,408,620, 9.717,519, 9,439,664, and 9,427,252.

However, none of the devices currently available is ideal for treating DVT or PE. The ideal thrombectomy device would be designed to retract hard and soft clots in DVT and PE patients in a single pass without trauma to the vessel. An essential aspect of a DVT / PE thrombectomy device is its effectiveness at removing obstructive thrombi, thereby achieving a rapid improvement in hemodynamics, and avoiding ischemic complications. The ideal device would allow rapid passage and advancement into veins and arteries, but must also filter distal thrombi. The device must be safe for the patient without causing damage to vascular structures, and blood loss during the procedure must be minimized. Only 3-5% of DVT and PE cases are treated today with mechanical clot removal devices. Currently, all devices for thrombectomy are costly. There is, therefore, an ongoing unmet need for new devices and approaches that can safely and reliably removal clots in DVT and PE patients.

### SUMMARY OF THE INVENTION

An intravascular thrombus retraction device includes: wires that are compressible into a compact cylindrical form within a catheter and are self-expandable into a wire mesh web with at least some parallel wires forming openings in the wire mesh sufficient to allow fluid passage and small enough to filter particles of at least 0.001 mm, a base of the wire mesh web connected to radial ring-shaped structure supporting and maintaining an opening in the base of the wire mesh and forming a thrombus capture volume, and the radial ring-shaped structure being compressible into the catheter and being self-expandable when free of compressive forces within the catheter to open up into an open, expanded, radial ring-shaped structure which maintains the opening in the opening in the base of the wire mesh. There are also multiple intermediate guide wires connected to and spaced about the ring-shaped structure; the multiple intermediate guide wires are connected to withdrawal guidewires extending into the catheter. It is preferred that at least some of the wires in the wire mesh have protrusions extending inwardly into the thrombus capture volume, at least some of the protrusions having a height less than a distance between the at least some parallel wires.

A method for performing a thrombectomy with this device is also disclosed in which the method is executed by:
a. inserting the device into a blood vessel having a thrombus;
b. advancing a distal end of the catheter of the device towards a thrombus;
c. deploying the compressed wire mesh and ring-shaped structure distally past the thrombus;
d. expanding the wire mesh and ring-shaped structure past the thrombus;
e. retracting the wire mesh and ring-shaped structure by applying tension to the withdrawal guidewires in a first retraction step; and
f. capturing the thrombus within the wire mesh during the first retraction step.

Cardiovascular disease may arise from accumulation of atheromatous material on the inner walls of vascular lumens. If a partially or completely occluded vessel provides blood to sensitive tissue such as the brain or heart, for example, serious tissue damage may result. Vascular deposits may restrict blood flow through an artery and can cause ischemia in the heart, legs, lungs, or brain, which may lead to pain, swelling, wounds that will not heal, amputation, stroke, myocardial infarction, and/or other conditions.

The present disclosure discloses a medical device capable of retracting clots from blood vessels using a collecting mechanism and aspiration, so that the retraction device and clot are withdrawn proximally through the guiding catheter out of the body. Deposits may be treated by drugs, bypass surgery, and atherectomy, including a variety of catheter-based approaches based on intravascular removal of deposits occluding a blood vessel. A catheter-based system may be utilized for removing a thrombus, wherein the catheter may be extended distal to a thrombus in a blood vessel wherein the thrombus is retracted from the vessel.

A limiting factor with available thrombectomy catheter devices is the difficulty to identify and treat hard thrombus. Current thrombectomy devices do not reliably break the thrombus away from the wall of the vessel. Current thrombectomy catheters are typically bulky and require manipulation towards the thrombus to avoid the risk of a distal embolism.

Basic aspiration catheters have a proximal end connected to a suction pump which causes fluid to enter the distal opening of the hollow lumen and travel to the proximal end of the lumen. Conventional aspiration catheters are typically threaded through a balloon guide catheter. In one exemplary procedure, the balloon of the guide catheter is inflated to occlude the vessel. The distal end of the aspiration catheter is typically advanced to the blood clot, with suction connected to the aspiration catheter to cause flow reversal.

One fundamental issue with thrombectomy catheters is that thrombotic burden can be highly variable. Mechanical catheters may have size constraints with respect to their use on larger thrombi. Aspiration devices have operational limits when the diameter of the catheter limits their use to small thrombi. Large thrombi on the other hand, will not pass into the catheter, which creates a risk of embolism. Since blood is extracted alongside the thrombus in the thrombectomy procedure, aspiration can potentially cause hemodynamic deterioration in patients with pulmonary-embolism-related shock. The flexibility and durability of aspiration catheter systems may thus limit their use.

Thrombi normally must deform to the inner diameter of the aspiration catheter. The applied vacuum may partially draw a thrombus into the distal opening of the aspiration catheter’s lumen, thereby deforming some of the thrombus to the catheter’s inner diameter. If the thrombus becomes lodged within the distal opening of the aspiration catheter, the only option is to pull the clot back through the balloon guide. Pieces of the clot can break off during movement. When the clot is drawn out from the patient, it is difficult to confirm that the entire thrombus was removed.

An aspiration system that increases the first-pass recanalization rate can be a useful metric. Prior art systems are often not able to react quickly enough to keep the distal end of the catheter from experiencing a positive pressure. Thus, a need exists to overcome the problems with recanalization systems, designs, and processes.

One aspect of the present disclosure is to provide a mechanical thrombectomy system that is flexible enough so that it can reliably and safely navigate blood vessels to a clot.

A second aspect of the present disclosure is to provide a mechanical thrombectomy device that can reliably entrap a soft or hard thrombus without fragmenting the thrombus or damaging the intima of the blood vessel. The innermost layer of tissue comprising the walls of blood tunica intima is the innermost layer of tissue comprising the walls of blood vessels. Although it is sometimes mistakenly referred to as the tunica interna, “intima” is the proper designation.

A third aspect of this disclosure is to provide a mechanical thrombectomy device that is biocompatible and compatible with standard medical catheters.

A fourth aspect of this disclosure is to provide a mechanical thrombectomy device that can safely and completely remove large clots of any density from the upper leg, pelvis, and lung.

A fifth aspect of the disclosure is to provide a mechanical thrombectomy device that reduces the risk of fragmentation and distal embolization when used in association with aspiration.

A sixth aspect of this disclosure is to provide an aspiration system that increases the first-pass recanalization rate during thrombus removal.

The current technology may further include a method using a unique thrombus retraction device. The invention may include removing a blood clot from a position within a blood vessel with a process of at least:
inserting a delivery catheter into a region within a blood vessel without passing the position of the blood clot, the delivery catheter comprising an exterior catheter, and a deployment catheter within a lumen of the delivery catheter;
the deployment catheter includes:
a) an interior catheter with a second lumen, and within the second lumen in linear orientation within the second lumen are three separate and distinct compressed and expandable mesh elements connected by at least two struts passing along a surface of a middle one of the compressed and expandable mesh elements;
b) the at least two struts having elastic memory sufficient to expand themselves and provide expansion forces on the three separate and distinct compressed and expandable mesh elements;
c) a most distal one of the three separate and distinct compressed and expandable mesh elements having an opening which faces towards the deployment catheter and the middle one of the three separate and distinct compressed and expandable mesh elements after the most distal one of the compressed and expandable mesh elements is deployed and expands;
d) a most proximal one of the three separate and distinct compressed and expandable mesh elements having an opening which faces away from the deployment catheter and towards the middle one of the three separate and distinct compressed and expandable mesh elements after the most proximal one of the compressed and expandable mesh elements is deployed and expands;
   the method may further include extending the deployment catheter past the clot while the exterior catheter does not pass the position of the blood clot;
   once a leading end of the deployment catheter has passed the position of the blood clot, deploying only the most distal one of the three separate and distinct compressed and expandable mesh elements past the position of the blood clot, while the middle one of and the most proximal one of the three separate and distinct compressed and expandable mesh elements remain between the clot and the interior catheter;
   the at least two struts expanding to expand all of the three separate and distinct compressed and expandable mesh elements;
   withdrawing the most distal one of the three separate and distinct compressed and expandable mesh elements towards the blood clot and supporting it on or within the opening which faces towards the deployment catheter and the middle one of the three separate and distinct compressed and expandable mesh elements, and bringing the most distal mesh element and the middle one of the mesh elements together, with the blood clot stabilized between the most distal mesh element and the middle mesh element;
   bringing the stabilized blood clot and the most distal mesh element and the middle one of the mesh elements towards the most proximal of the three separate and distinct compressed and expandable mesh elements;
   at least partially withdrawing the deployment catheter into the delivery catheter; and
   withdrawing the delivery catheter from the blood vessel along with the blood clot.

The method may further include bringing the stabilized blood clot and the most distal mesh element and the middle one of the mesh elements towards the most proximal of the three separate and distinct compressed and expandable mesh elements;
at least partially withdrawing the deployment catheter into the delivery catheter; and
withdrawing the delivery catheter from the blood vessel along with the blood clot are performed sequentially.

### BRIEF DESCRIPTION OF THE DRAWINGS

This invention may be more completely understood with respect to the following description of various embodiments. While the disclosure is amenable to various modifications and alternative forms, specifics have been shown by way of example in the drawings and will be described in detail.

FIGS. 1A-G show serial views of the intravascular thrombectomy catheter during isolating, capturing, and removing blood clots from a blood vessel.

FIG. 1A shows a schematic view of the catheter device for retracting intravascular thrombotic material.

FIG. 1B shows the intravascular thrombectomy catheter device with its distal tip extending beyond the distal edge of the thrombus.

FIG. 1C shows the thrombectomy catheter device collapsed inside the retraction catheter with the guidewire removed.

FIG. 1D shows the thrombectomy device with catheter components deployed.

FIG. 1E shows the retraction catheter being pulled with the thrombectomy device over the thrombus.

FIG. 1F shows the collecting basket being positioned to collect the thrombus.

FIG. 1G shows the collecting basket deployed over the thrombus.

FIG. 1H is a cross-sectional illustration of a multi-lumen catheter with an inserted optical fiber that can be used to measure an optical signal in a thrombus in an artery.

FIG. 1I is a schematic of the brain of a patient illustrating a thrombus retraction procedure in which fluoroscopic images may be acquired and stored in memory by an MRI-based tracking system.

FIG. 2 is a schematic view of the thrombectomy catheter device showing the arrangement of the struts and the ring segments in relation to a thrombus.

FIG. 3 is a schematic view of the distal embolic protection component of the thrombectomy device.

FIG. 4 is a schematic of a tracking system to evaluate clot treatment in individual patients.

FIG. 5 is a schematic view of the major veins in the upper leg, pelvis, and thorax involved in DVT and PE.

FIG. 5B shows the use of image guidance to guide the thrombectomy device through the vasculature.

FIG. 6 is a schematic view of a custom-made handle to advance of the DVT / PE thrombectomy device.

FIG. 7 show the components of an aspiration device that can be added to the manifold attached to the hub of the guiding catheter or the collecting catheter.

FIG. 8 shows a mesh wire web having different protruding elements, protrusions, dots, or gripping elements that are on the wire, generally facing inward (towards where a clot or the thrombus would be in contact with the wire).

FIG. 8A shows a partially extended, unexpanded distal mesh from the delivery catheter.

FIG. 8B shows a fully extended, expanded distal mesh and expander mesh and three struts extending from the delivery catheter.

FIG. 9 shows a side view of a shaped distal mesh element capturing a clot.

FIG. 9A and FIG. 9B

FIG. 10 shows a fully deployed capture system.

FIG. 11 shows three different aspects or perspectives of the deployed ring-shaped structure’s four segments.

FIG. 12 shows a different perspective of the deployed ring-shaped structure segments of FIG. 11.

FIG. 13 shows a pre-deployed 13A catheter delivery system and deployed 13B catheter delivery assembly comprising a catheter, single retraction guidewire, and multiple dual wire element distal retraction guidewires.

FIG. 14A shows a pre-deployed advanced catheter delivery system with three deployable, expandable elements with at least three struts within the deploying catheter which are also carried, along with deployable, expandable elements.

FIG. 14B shows a partially deployed advanced catheter delivery system with three deployed, expandable elements.

FIG. 14C shows a fully deployed advanced catheter delivery system with three deployed, expandable elements still not expanded.

FIG. 14D shows a fully deployed advanced catheter delivery system with three deployed, expandable elements.

FIG. 15 shows the distal mesh element and the intermediate mesh element with a clot therebetween. The intermediate mesh element has an additional expandable element that can impact the clot within the expanded distal mesh element.

FIG. 15A shows an axial view of a blood vessel with a clot attached more against a single wall.

FIG. 15B shows a perspective view of the blood vessel and clot of 15A with the deployment device inserted and positioned past the clot, but no expandable elements deployed.

### DETAILED DESCRIPTION OF THE INVENTION

This disclosure provides design, material, manufacturing method, and use alternatives for medical devices and systems. The device for removal of intravascular thrombus removal comprises accessing a venous blood vessel of a patient in which a retraction catheter is inserted to a site of clot. An aspiration catheter with wall-mounted suction may be attached to remove a vascular obstruction with one pass. Aspiration may be applied to the guiding or collecting catheters to decrease embolization of clot fragments.

An intravascular thrombus retraction device includes: wires that are compressible into a compact cylindrical form within a catheter and are self-expandable into a wire mesh web with at least some parallel wires forming openings in the wire mesh sufficient to allow fluid passage and small enough to filter particles of at least 0.001 mm, a base of the wire mesh web connected to radial ring-shaped structure supporting and maintaining an opening in the base of the wire mesh and forming a thrombus capture volume, and the radial ring-shaped structure being compressible into the catheter and being self-expandable when free of compressive forces within the catheter to open up into an open, expanded, radial ring-shaped structure which maintains the opening in the opening in the base of the wire mesh. There are also multiple intermediate guide wires connected to and spaced about the ring-shaped structure, the multiple intermediate guide wires are connected to withdrawal guidewires extending into the catheter. It is preferred that at least some of the wires in the wire mesh have protrusions extending inwardly into the thrombus capture volume, at least some of the protrusions having a height less than a distance between the at least some parallel wires.

An alternative intravascular thrombus retraction device includes: wires that are compressible into a compact cylindrical form within a catheter and are self-expandable into a wire mesh web with at least some parallel or helical wires forming mesh openings in the wire mesh sufficient to allow aqueous fluid passage and small enough to filter particles of at least 0.001 mm or thrombus particles having a size which is recognized as having potentially harmful effects in at least the smaller blood vessels in the brain. A base of the wire mesh web is connected to radial ring-shaped structure supporting and maintaining an opening in the base of the wire mesh and forming a thrombus capture volume within the wire mesh. The radial ring-shaped structure could in theory be a single continuous element having an elastic memory that is the radial ring shape, but for purposes of construction of the device, a radial ring element having more than two bend or flex points, of having pivots, rotating connections, or segmented elements that allow for easier and more shapely compression may be used. The radial ring-shaped structure is as described compressible into a thin roughly cylindrical shape within the catheter and is self-expandable when free of compressive forces within the catheter to open up into an open, expanded, radial ring-shaped structure which maintains the opening in the opening in the base of the wire mesh. There are also multiple intermediate guide wires connected to and spaced about the ring-shaped structure, the multiple intermediate guide wires are connected to withdrawal guidewires extending into the catheter. It is preferred that at least some of the wires in the wire mesh have protrusions extending inwardly into the thrombus capture volume, at least some of the protrusions having a height less than a distance between the at least some parallel wires.

The wire may be a non-thrombogenic metal and the protrusions have a height of less than 0.001 mm. Because of the relatively short time duration of the device within a blood stream, there may be a tolerable range of materials that can be used if they are non-thrombogenic within the time frame of the surgery. The protrusions may be elements, bumps, rods, and the like extending from surfaces of the wires and the protrusions may have concave, convex, flat, curvilinear, or pointed tips.

The device may include two catheters, a first catheter containing the wire mesh and ring-shaped structure in a compressed, non-expanded state, and a second catheter containing a compressed and expandable collection receptacle, the collection receptacle positioned within the second catheter such that upon release from the catheter, the collection receptacle expands to provide an opening in an opposed position with respect to the opening in the base of the wire mesh of a released and expanded wire mesh and ring-shaped structure.

The current technology further includes a method using a unique thrombus retraction device. The invention may include removing a blood clot from a position within a blood vessel with a process of at least:
inserting a delivery catheter into a region within a blood vessel without passing the position of the blood clot, the delivery catheter comprising an exterior catheter, and a deployment catheter within a lumen of the delivery catheter;
the deployment catheter includes:
   a) an interior catheter with a second lumen, and within the second lumen in linear orientation within the second lumen are three separate and distinct compressed and expandable mesh elements connected by at least two struts passing along a surface of a middle one of the compressed and expandable mesh elements;
   b) the at least two struts having elastic memory sufficient to expand themselves and provide expansion forces on the three separate and distinct compressed and expandable mesh elements;
   c) a most distal one of the three separate and distinct compressed and expandable mesh elements having an opening which faces towards the deployment catheter and the middle one of the three separate and distinct compressed and expandable mesh elements after the most distal one of the compressed and expandable mesh elements is deployed and expands;
   d) a most proximal one of the three separate and distinct compressed and expandable mesh elements having an opening which faces away from the deployment catheter and towards the middle one of the three separate and distinct compressed and expandable mesh elements after the most proximal one of the compressed and expandable mesh elements is deployed and expands;
      the method may further include extending the deployment catheter past the clot while the exterior catheter does not pass the position of the blood clot;
      once a leading end of the deployment catheter has passed the position of the blood clot, deploying only the most distal one of the three separate and distinct compressed and expandable mesh elements past the position of the blood clot, while the middle one of and the most proximal one of the three separate and distinct compressed and expandable mesh elements remain between the clot and the interior catheter;
      the at least two struts expanding to expand all of the three separate and distinct compressed and expandable mesh elements;
      withdrawing the most distal one of the three separate and distinct compressed and expandable mesh elements towards the blood clot and supporting it on or within the opening which faces towards the deployment catheter and the middle one of the three separate and distinct compressed and expandable mesh elements, and bringing the most distal mesh element and the middle one of the mesh elements together, with the blood clot stabilized between the most distal mesh element and the middle mesh element;
      bringing the stabilized blood clot and the most distal mesh element and the middle one of the mesh elements towards the most proximal of the three separate and distinct compressed and expandable mesh elements;
      at least partially withdrawing the deployment catheter into the delivery catheter; and
      withdrawing the delivery catheter from the blood vessel along with the blood clot.

The method may further include bringing the stabilized blood clot and the most distal mesh element and the middle one of the mesh elements towards the most proximal of the three separate and distinct compressed and expandable mesh elements;
at least partially withdrawing the deployment catheter into the delivery catheter; and
withdrawing the delivery catheter from the blood vessel along with the blood clot are performed sequentially.

The method may further include bringing the stabilized blood clot and the most distal mesh element and the middle one of the mesh elements towards the most proximal of the three separate and distinct compressed and expandable mesh elements;
at least partially withdrawing the deployment catheter into the delivery catheter; and
withdrawing the delivery catheter from the blood vessel along with the blood clot are performed contemporaneously.

The method may have a surface of the middle mesh element conformed against a surface of the most distal mesh element, and particularly against the opening in the most distal mesh element.

The method may be practiced wherein there are at least three struts with elastic memory deployed from the deployment catheter. The elastic memory bows the struts outwardly, away from a central axis of the inner catheter of the deployment catheter and exerting expanding forces on at least some, if not all of the three separate and distinct compressed and expandable mesh elements, and always the middle of the three mesh elements.

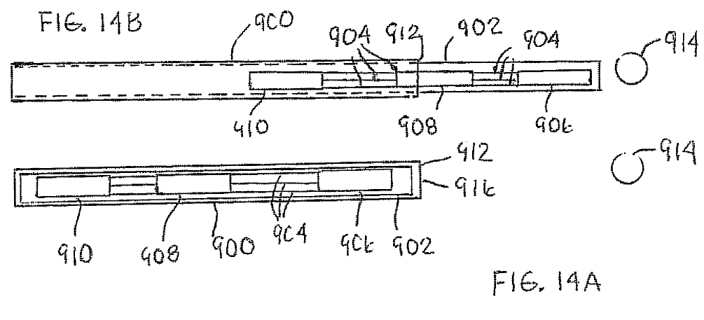

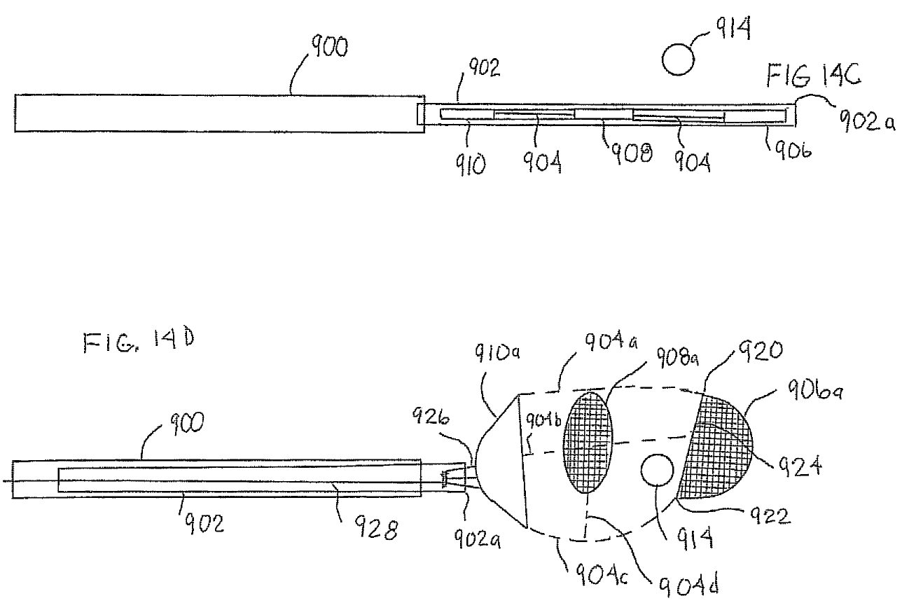

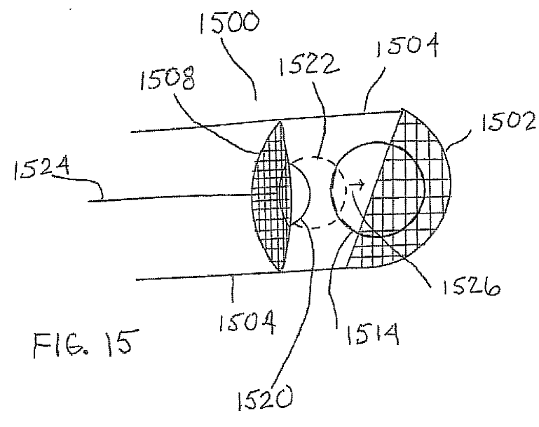

The method may be practiced wherein the opening in the most distal one of the mesh elements forms a surface that is not orthogonal to surfaces within the blood vessel. The opening may be orthogonal to both opposed surfaces of a blood vessel, but may also be angled between 5 degrees to 90 degrees (measured by a closest point or contact point and referred to herein as the “contact point,” even if not in contact, between one edge of the opening and one of the opposed surfaces. Preferably this angle (as measured from a range including the most obtuse angles between the contact point (e.g., 922) and an opposed portion of the opening 924). The opening may also be curved, so that when the opening is viewed from a side view, element 920 in FIG. 14D may be straight, concave or convex. The angling at contact point 922, which may be forward leaning or rearward leaning, provides two apparent improvement functions in the practice of the method. It may act to assure a faster and more complete expansion of the distal compressed mesh element 906 and it may act to assist in scooping in thrombi or emboli adjacent surfaces of the blood vessel.

The device may have the ring-shaped structure include or be attached to struts which place expanding or restraining force on the ring-shaped structure to maintain the opening in an expanded and open position.

The device may also or alternatively have the collection receptacle include or be attached to struts which place expanding or restraining force on the opening in the opposed position to maintain the opening in the opposed position in an expanded and open position.

A method of capturing a thrombus within vasculature may include: comprising providing the above described intravascular thrombus retraction device, which may alternatively be characterized as wires that are compressible into a compact cylindrical form within a catheter and are self-expandable into a wire mesh web with at least some parallel wires forming openings in the wire mesh sufficient to allow fluid passage and small enough to filter particles of at least 0.001 mm, a base of the wire mesh web connected to radially ring-shaped structure supporting and maintaining an opening in the base of the wire mesh and forming a thrombus capture volume, the ring-shaped structure being compressible into the catheter and being self-expandable when free of compressive forces within the catheter to open up into the open, expanded ring-shaped structure, maintaining the opening in the opening in the base of the wire mesh, multiple intermediate guide wires are connected to and spaced about the ring-shaped structure, the multiple intermediate guide wires are connected to withdrawal guidewires extending into the catheter, at least some of the wires in the wire mesh having protrusions extending inwardly into the thrombus capture volume, at least some of the protrusions having a height less than a distance between the at least some parallel wires; the method comprising:
a) inserting the device into a blood vessel having a thrombus;
b) advancing a distal end of the catheter of the device towards a thrombus;
c) deploying the compressed wire mesh and ring-shaped structure distally past the thrombus;
d) expanding the wire mesh and ring-shaped structure past the thrombus;
e) retracting the wire mesh and ring-shaped structure by applying tension to the withdrawal guidewires in a first retraction step; and
f) capturing the thrombus within the wire mesh during the first retraction step.

The wire may be composed of a non-thrombogenic metal and the protrusions have a height of less than 0.001 mm, and during the first retraction step, the protrusions engage and grasp a surface of the thrombus.

The present disclosure further relates to a method of treating DVT and PE in the peripheral vasculature of a patient. The method includes providing a thrombectomy device that can be tubular and is formed of a braided filament mesh structure. The mesh structure can have a proximal end of the attached to a distal end. The invention includes advancing a catheter with the thrombectomy device through a vascular thrombus in a venous vessel. A shaft extends through the catheter and a distal end is coupled to a proximal end. The method includes deploying the thrombectomy device from the catheter from a constrained configuration to an expanded configuration. In some embodiments, the thrombectomy device engages at least a wall of the venous vessel distally past the thrombus at full expansion. The method includes retracting the thrombectomy device proximally to separate a portion of the thrombus from the venous vessel wall while the mesh structure captures the thrombus. The method includes withdrawing the thrombectomy device from the patient to remove the thrombus from the venous vessel.

Advancing the thrombectomy device includes inserting the catheter into the venous vessel until a radiopaque distal tip of the catheter is distally past the thrombus. In some embodiments, deploying the thrombectomy device from the constrained configuration to the expanded configuration includes advancing the shaft distally until the thrombectomy device is beyond a distal end of the catheter. Deploying the thrombectomy device further includes determining a position of the thrombectomy device with respect to the catheter via imaging of a first radiopaque marker located on the catheter and a second radiopaque marker located on at least one of the shaft or mesh structure.

The vascular thrombectomy device is added into the mesh structure by entering the expandable tubular portion via at least an aperture located at the proximal end of the self-expanding stent. The method includes inserting the catheter into the venous vessel through an access site, which is a popliteal venous site, a femoral venous site, or an internal jugular venous site. The venous vessel has a diameter of at least 5 millimeters and may include a femoral vein, an iliac vein, a popliteal vein, a posterior tibial vein, an anterior tibial vein, or a peroneal vein.

The method further includes: percutaneously accessing the venous vessel of the patient with an introducer sheath through an access site into the venous vessel of the patient, advancing a distal end of the introducer sheath to a position proximal of the thrombus, and inserting the catheter through a lumen of the introducer sheath so that a distal tip of the catheter is distally past the thrombus.

Withdrawing the thrombectomy device from the patient includes: retracting the thrombus extraction device relative to the introducer sheath until an opening is within the self-expanding stent, collapsing the stent portion and mesh structure so as to compress the thrombus, retracting the stent portion and mesh structure into the introducer sheath, and removing the thrombectomy device from the introducer sheath.

The method may further include extruding at least some of the thrombus through the distal portion of the expandable tubular portion and capturing a part of the thrombus in the self-expanding funnel or further compressing the thrombus through a mesh of the self-expanding funnel. The method may further includes aspirating the thrombus through an aspiration port connected to a proximal end of the introducer sheath.

One aspect of the present disclosure relates to a method of treating DVT in a peripheral vasculature of a patient to include percutaneously accessing a venous vessel of a patient with an introducer sheath through a popliteal vein site; and inserting a catheter with a thrombectomy device through a lumen of the introducer sheath so that the catheter is distally past the thrombus.

In some embodiments of the invention, a proximal end of the mesh structure may be attached to a distal end of the fenestrated structure. The thrombectomy device may be deployed from a constrained configuration to an expanded configuration by advancing a shaft distally until the stent portion of the thrombectomy device is beyond the distal end of the catheter.

One aspect of the present invention relates to a removal of thrombus from an artery or a vein of a patient by providing a thrombectomy device with a net-like filament mesh structure; advancing with the thrombectomy device through a thrombus, and deploying the thrombectomy device to engage a wall of the blood vessel. Retracting the thrombectomy device to separate a portion of the thrombus from the vessel wall and to capture the portion of the thrombus within the net-like mesh structure to remove thrombus from the patient.

In the method of the invention, fluoroscopically monitoring deployment of the thrombectomy device beyond first radiopaque marker located on the catheter relative to a second radiopaque marker located on the thrombectomy device. In some embodiments, the thrombus is located in the peripheral vasculature of the patient and the blood vessel has a diameter of at least 5 millimeters and includes at least one of the following: a femoral vein, an iliac vein, a popliteal vein, a posterior tibial vein, an anterior tibial vein, or a peroneal vein. In some embodiments of the invention, the method includes aspirating or infusing a thrombolytic agent into or from the blood vessel before, during, or after thrombus extraction.

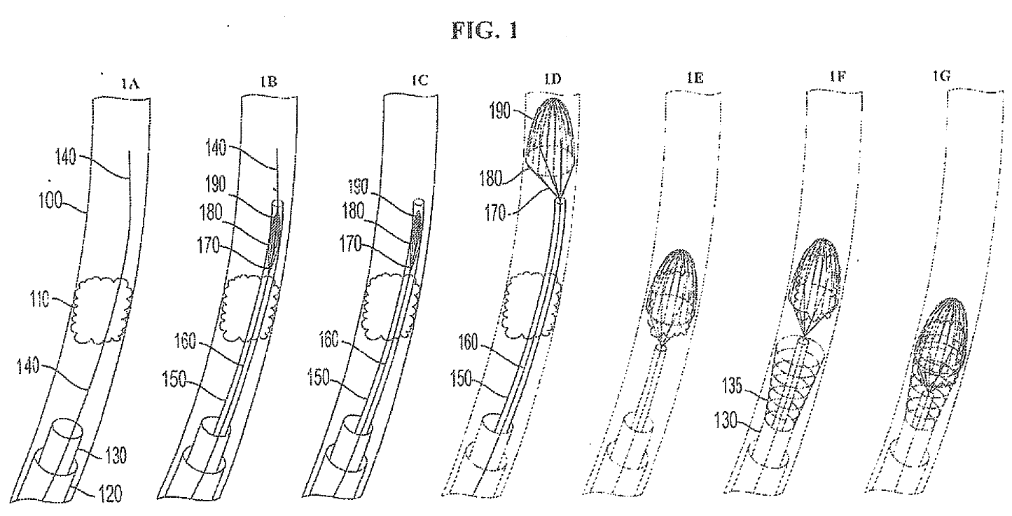

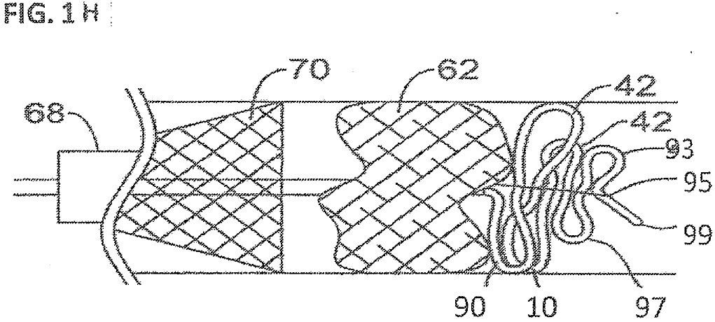

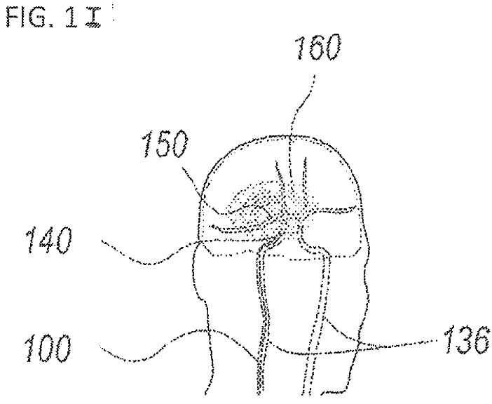

FIGS. 1A-G depict steps for the mechanical thrombectomy device in a blood vessel 100 with a thrombus 110. A guiding catheter 120 can be positioned by transluminal catheter delivery within the lumen of the blood vessel 100 proximal to the thrombus using image-guided techniques. A retraction catheter 130 can pass through the guiding catheter 120 and may be positioned just below the proximal aspect of clot 110. As shown in FIG. 1A, a guidewire 140 can be placed proximal or distal to the thrombus 110 to be used to guide the collecting or retraction catheter 130. The retraction catheter 130 can then pass through guiding catheter 120 and over guidewire 140 to a position with its distal tip placed distal to the distal edge of the thrombus 110 as shown in FIG. 1B. The thrombectomy device with retracting wire 140, struts 170, ring structure 180 and web 190 can be passed through the retraction catheter or may be pre-loaded inside the retraction catheter 130. As shown in FIG. 1C, the guidewire 160 can be removed. The thrombectomy device can be deployed by manipulating the retraction wire 160 as shown in FIG. 1D. As shown in FIG. 1E, the retraction catheter 150 can be positioned proximally with the thrombectomy device pulled down over thrombus 110 in FIG. 1A. As shown in FIG. 1F, the collecting basket 135 may be deployed from the collecting catheter 130 to collect the thrombus as shown in FIG. 1G.

When possible, the entire thrombus 110 may be pulled into the guiding catheter 120 and removed from the body, leaving the guiding catheter 120 in place. If the clot is too large to be pulled into and through the guiding catheter 120, aspiration may be applied to the guiding catheter or the collecting catheter 130, wherein a catheter connected to an aspiration system can be hooked to the flushing system for the guiding catheter via a 3-way stopcock. Aspiration can be usefully added when applied to the clot that has been pulled into the collecting catheter 130 to make it smaller for removal through the guiding catheter 120.

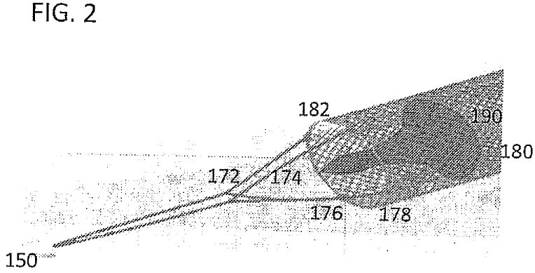

FIG. 2 is a schematic view of the thrombectomy device showing the arrangement of the struts 172, 174, 176, 178 and the ring segment 182. The device uses a web structure with retraction wires to retract thrombus 190. The device includes expandable struts having a closed compact configuration 184 and an open expanded configuration 186. In an optional embodiment, additional collection features such as cactus-claws, end-hooks and hook-type imaging may be included. In some embodiments, these features can be formed from various metals or alloys such as Nitinol, platinum, cobalt-chrome alloys, 35N LT, Elgiloy™, stainless steel, tungsten, or titanium.

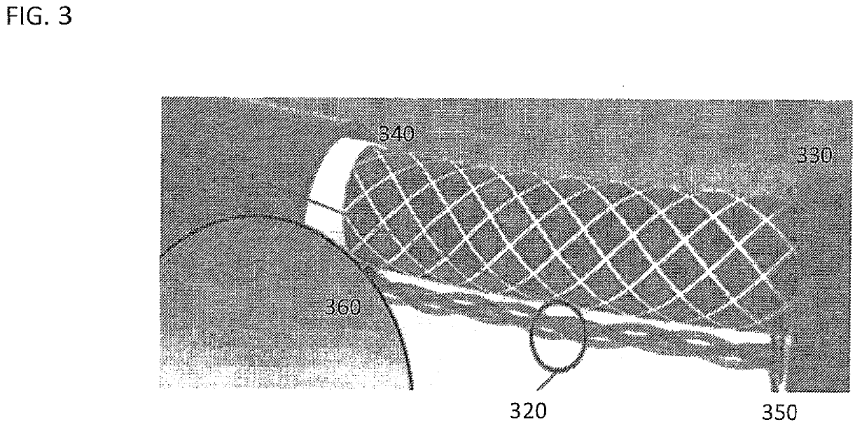

In one embodiment of the disclosure shown in FIG. 3, fluoroscopically visible markers 188 are applied to the retraction ring, the retraction wire, and the collecting basket 180 and to the tip of the guiding catheter 150 to facilitate localization of all components. Some examples of radiopaque materials include gold, platinum, palladium, tantalum, tungsten alloy, and polymer material loaded with a radiopaque filler. The thrombectomy device may also be made from a metal, metal alloy, polymer, a metal-polymer composite, ceramics, or other suitable material including, such as304V, 304L, and 316LV stainless steel; mild steel; nickel-titanium alloy such as linear-elastic and/or super-elastic nitinol; and nickel-chromium-molybdenum alloys.

In another embodiment, the device can be equipped with imaging sensors 350, or with sensors measuring physiological parameters 360 such as pressure, temperature, and oximetry. In one embodiment, FOSS technology facilitates the visualization of thrombectomy catheters 330 and wires 340 without the need for fluoroscopy. In addition to reducing the need for X-ray exposure of the patient and medical personnel, the FOSS technology also enables more detailed views of device positioning. In an exemplary embodiment, optical fibers are embedded in the device and equipped with Fiber Bragg Gratings, which enables the determination in 3 dimensions of the shape and position of the catheters and wires in real-time and with high accuracy. The shape and position of the catheters and wires can then be superimposed on roadmap views of the vasculature and pathology.

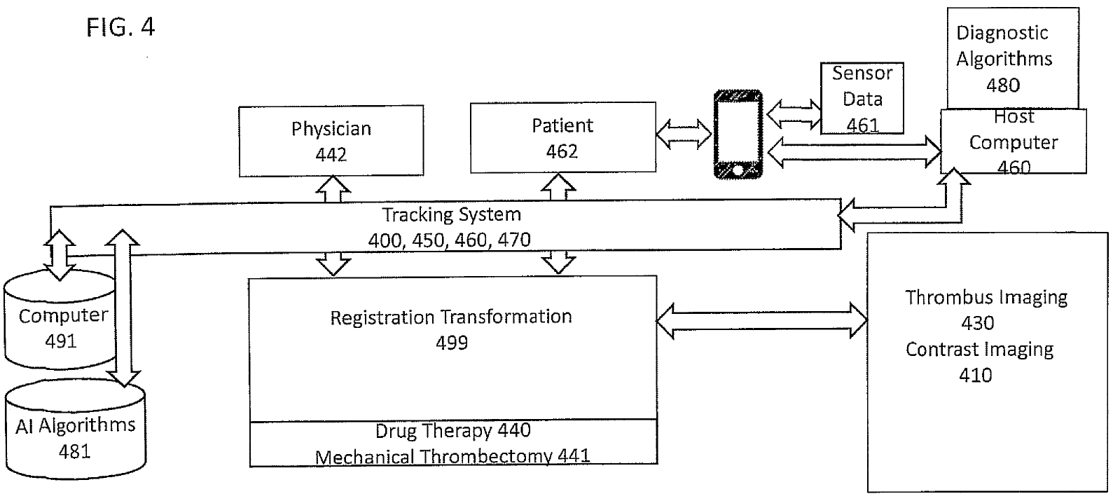

As shown in FIG. 4, sensors 461 may be connected to a host computer 460, wherein diagnostic algorithms 480 can be used to evaluate treatment plans for individual patients 462 and to actively modify existing treatment plans by physicians 442. by the host computer 460. Observation of imaging changes 499 can be clinically A computer-based 491 tracking system 400 can be used in patient studies to investigate clot composition with respect to the age 450, composition 460 and size of a thrombus 470. During formation of a thrombus, characteristic alterations in fluorescence contrast imaging 410 and thrombus imaging 430 may be registered useful in evaluating the potential utility of various alternative therapeutic interventions, such as, for example, drug thrombolytic therapy 440 and mechanical thrombectomy 441. Imaging 410, 430 may be used to guide the thrombectomy device through the vasculature 520 through under manual control of a host computer 460. Since branching of the main pulmonary arteries may be anatomically complex, it may be difficult to locate and catheterize an occluded vessel 500 when using single plane fluoroscopy 410.

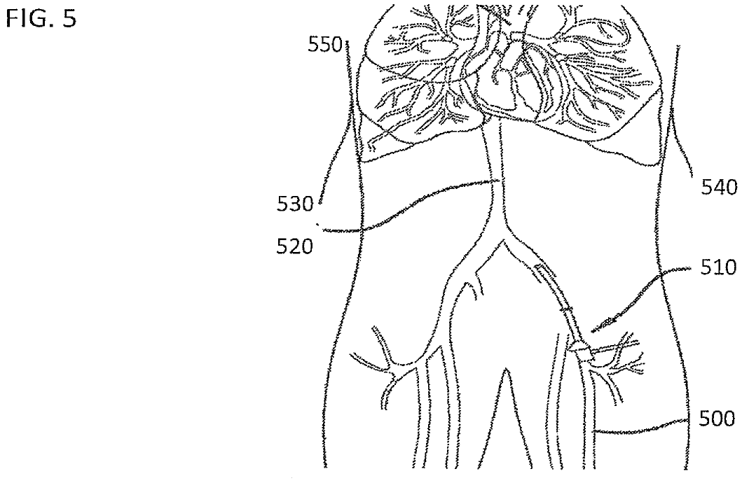

As shown in FIG. 5, image guidance can be used to guide the thrombectomy device through the vasculature 500 toward the correct location 530, 540, to manipulate various functions of the device 510, and to manage the retraction of the thrombus (not shown) PE is considered to be part of the same continuum of disease as DVT with over 95% of emboli originating in the legs. Mechanical clot removal, as disclosed in the present disclosure, is relatively rapid compared to the use of thrombolytics. Pulmonary emboli, particularly those in the proximal aspects of the pulmonary arteries, can be quite large requiring a larger retriever catheter and wires than for most DVT cases. Biplane fluoroscopic systems such as those used for cerebral angiography and intervention can be used to improve the catheterization process.

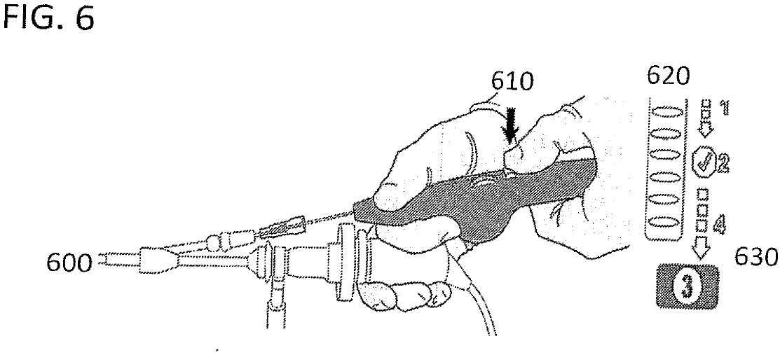

The handle 600 (FIG. 6) may have a proximal end portion and a distal end portion. The distal end portion of the handle which may be connected or attached to the proximal shaft. In some embodiments, an adaptor may facilitate a connection between the handle and the proximal shaft. The handle may be formed from a polymer material, a metal material, a combination of metal material and a polymer material, and/or one or other suitable materials. Further, the handle may be formed with a suitable forming technique including machining, molding, grinding, injection molding, and laser cutting.

In one embodiment shown in FIG. 6, the device has a custom-made handle 600, in which an operator can engage a thumb engaging surface of the device 610 to transmit movement to the device through a compressed coil spring 620, wherein the operator is able to apply a calibrated force 630 to the device. The calibrated forces may be spaced a predetermined linear distance, to include detents, cut-outs, recesses, spacings, notches, indents, bumps, protrusions and/or other features. As shown in FIG. 6, the calibration forces may be linearly drawn as it is moved one or more predetermined distances in the longitudinal direction. In one example, the adjustment member may include a portion having a protrusion to include a cut-out, recess, spacing, notch, indent, and/or other formations to facilitate engaging the restrictions in or on the handle.

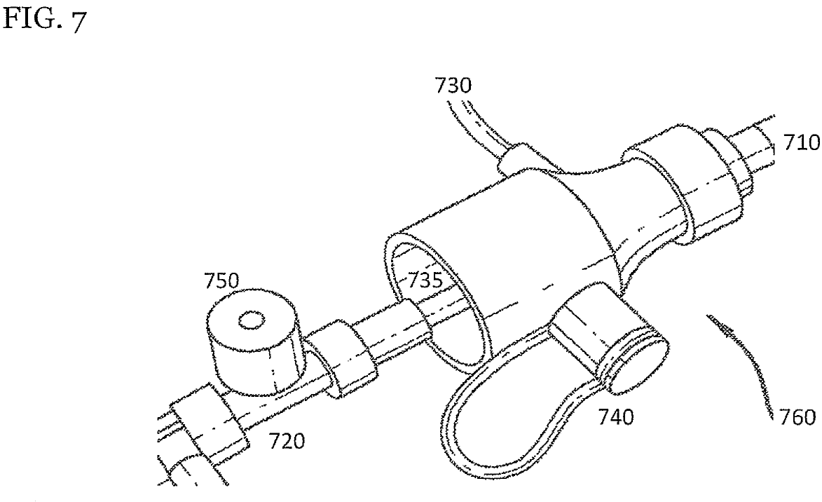

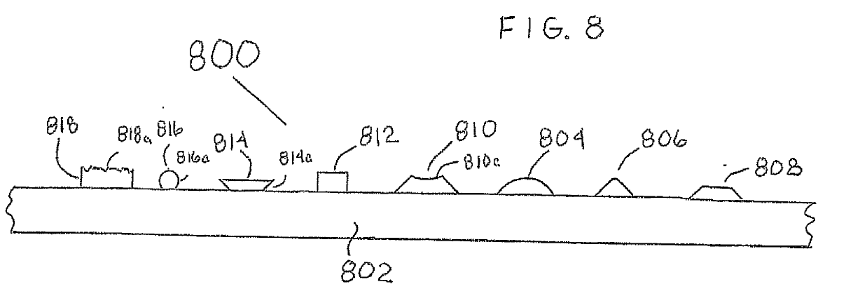

As shown in FIG. 7, a catheter 710 connected to an aspiration device 720 can be added to the manifold attached to the hub of the guiding catheter 730 or the collecting catheter 735 so that aspiration can be applied to the entire system to facilitate thrombus retraction, and prevent fragmentation and embolization of fragments through the guiding catheter 730. Aspiration coupled with mechanical thrombectomy 740 can thereby assist in the retraction of large clots. An aspirational system attached to the manifold of the guiding catheter may help reduce the size of the clot within the collecting device 735 to facilitate removal of the thrombus. Aspiration can also reduce the potential for distal embolization as the clot is manipulated.

The entire thrombus may be pulled into the guiding catheter 120 and removed from the body, leaving the guiding catheter 120 in place. If the clot 110 is too large to be pulled into and through the guiding catheter 120, aspiration may be applied to the guiding catheter or the collecting catheter 130 , wherein a catheter connected to an aspiration system can be hooked to the flushing system for the guiding catheter via a 3-way stopcock 750. Aspiration 760 can thus be usefully added when applied to the clot that has been pulled into the collecting device 4 to make it smaller for removal through the guiding catheter 120.

A method of treating deep vein thrombosis and pulmonary embolisms may include accessing a venous vessel of a patient, wherein a retraction catheter containing a clot treatment device is inserted into the venous circulatory system to a site of clot, wherein an aspiration catheter in inserted with wall-mounted suction attached to its inflow port, wherein the aspiration component can remove clot and other debris, and, wherein complete removal of both soft and hard components of a vascular obstruction is completed with one pass within in ninety percent of cases.

A device that may be used in the method may include a device equipped with a collecting mechanism in the form of a collecting catheter that passes over the retraction catheter, and that is equipped with a collecting structure that can be deployed when moving the collecting catheter beyond the end of the guiding catheter, surrounding the object when the object is extracted using the retraction catheter.

The method may further include accessing a venous vessel, inserting into retraction catheter into vessel, and restoring blood flow using the clot retraction device.

An alternative multi-lumen, multi-functional catheter system may include a plurality of axial lumens, wherein at least one physiological measuring device is present within a clot retraction catheter, wherein said physiological measuring device is connected to a host computer which is equipped for receiving information regarding DVT and PE treatment plans, wherein the host computer contains a treatment planning and therapy algorithm for individual DVT and PE patients, and, wherein the host computer signals the operator to actively modify the existing treatment plan as the therapy algorithm progresses.

A thrombectomy catheter comprising: an elongate flexible catheter body having a proximal end, a distal end and a central lumen extending longitudinally through the catheter body, wherein the catheter comprises a catheter with a variable durometer outer jacket, wherein the catheter wall thickness ratio of the inner diameter to the outer diameter is 0.80 or higher, wherein the tensile strength of the catheter is higher than 2 lbs.

Another device for removing blood clots may include an intravascular catheter having a distal end and a proximal end, the catheter having an inner lumen and an outer lumen, wherein an aspiration pump is attached to the proximal end of the catheter, and a mechanically actuated positive displacement powered by a rotating motor, wherein the motor rotates at a speed below 2000 RPM when driving the aspiration pump and wherein the speed of the motor is cycled at a frequency below 10 Hz.

Another method of treating deep vein thrombosis in a peripheral vasculature of a patient may include: percutaneously accessing a venous vessel of a patient with an introducer sheath through an access site into the venous vessel of the patient; inserting a catheter constraining a thrombectomy device through the lumen of the introducer sheath so that a distal tip of the catheter is distally past a portion of the thrombus ; deploying the thrombectomy device from a constrained configuration to an expanded configuration, wherein the thrombectomy device is in an expanded state between about 20 degrees and about 50 degrees; and, removing the thrombectomy device from the patient.

It will be apparent to those skilled in the art that various modifications and variations can be made in the disclosed systems and processes. It is intended that the specification and examples be considered as exemplary only.

Thrombus in the vasculature includes a range of morphologies and consistencies. Typically, older thrombus material contains a higher percentage of fibrin, making it less compressible with a harder outer surface that makes it more difficult to ensnare or aspirate than more acute thrombus which is softer. Current mechanical thrombectomy devices may not penetrate the surface of a hard fibrin-rich thrombus or produce sufficient force to grip the thrombus. It can be very difficult to aspirate a hard thrombus without first breaking it into pieces, which could then embolize into distal branches. During thrombectomy, 75-85% of thrombi can be removed using current devices, such as stent-retrievers and aspirators. However, the remaining 15-25% of intravascular thrombus cannot be easily removed by mechanical devices because the thrombus is hard.

CT and fluoroscopy imaging cannot typically identify the composition of intravascular thrombus, which may vary from relatively hard to relatively gel-like and soft. An obstructing thrombus in a blood vessel of the brain can be a medical emergency caused by occlusion of blood vessels to the brain or within the brain. Although an ischemic event can occur anywhere in the vascular system, the carotid artery bifurcation and the origin of the internal carotid artery are the most frequent sites for thrombotic occlusions of cerebral blood vessels.

Methods for imaging thrombus are reviewed in the present disclosure. As used herein, an imaging technology may include, positron-emission tomography, single photon emission computed tomography, magnetic resonance imaging, optical imaging, ultrasound, photoacoustic imaging, computed tomography, or near-infrared fluorescence-imaging.

FIG. 1H is a cross-sectional view of a catheter 10 in an artery adjacent to intravascular thrombus 62. In an exemplary embodiment of the disclosure, an optical fiber 95 is used to measure an optical signal collected from the thrombus 62, which is then output to an MRI-based tracking system 95. The catheter 10 may be advanced to the thrombus 62 to illuminate the thrombus 62, wherein an optical measurement unit 70 enables identification of the optical signal 42 of the thrombus 62 which reflects the content of the thrombus 62. A detector in the MRI system 99 converts the optical signal 42 into an electrical analog signal, and an analog-to-digital conversion circuit 93 digitizes the signal to the MRI tracking system 95 for analysis.

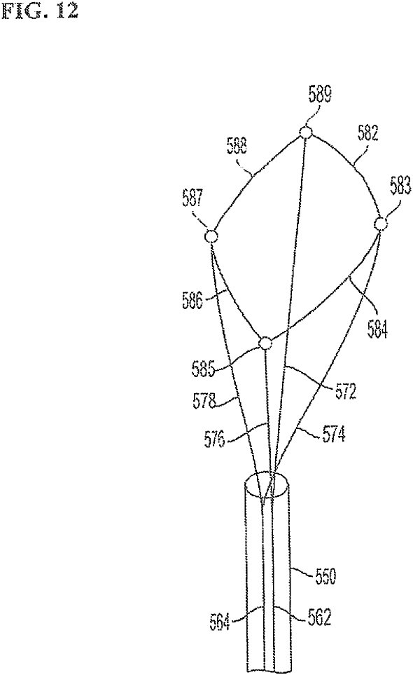

In one embodiment of the disclosure shown in FIG. 11, a schematic of the brain of a patient illustrates a thrombus retraction procedure 100 in which fluoroscopic images 136, 150, 160 may be acquired and stored in memory in a host computer for subsequent retrieval by an MRI-based tracking system 140.

A method for visualizing thrombus in an artery includes a wavelength-specific reflector being advanced to traverse the thrombus, wherein the incident light is selectively reflected at the diagnostic wavelength after interacting with the thrombus, wherein passing the optical signal through the thrombus increases an optical attenuation signal compared with a single pass, wherein the host computer analyzes transmitted optical signals, and, wherein the host computer identifies whether the thrombus is hard or soft based on the wavelength signal.

In the method, an optical fiber is adapted to allow light to interact with the thrombus, wherein hard thrombus absorbs less light than thrombus, and, wherein the MRI system can establish the composition of the thrombus based on the optical attenuation of the thrombus.

A device for tracking thrombus in a patient’s vasculature may include a measuring device connected to a host computer that can evaluate thrombus retraction, wherein the device is equipped with both optical sensors and imaging sensors, wherein the host computer contains a therapy algorithm for individual patients, and wherein the host computer can actively modify thrombus retraction as the therapy algorithm progresses.

The above device may have the host computer determine the thrombus composition based its optical transmission, and the MRI can be used to evaluate whether thrombus composition reduces its susceptibility to recombinant tissue plasminogen activator. The host computer may determine retraction routes, speed, and status of thrombus for individual patients.

A method for tracking thrombus in the vasculature comprising analyzing the intensity of an optical signal from a sensor in a catheter positioned in a blood vessel of a patient may include using an optical signal from the sensor is attenuated by thrombus, wherein an MRI-based host computer tracks the thrombus by analyzing the measured signal attenuation, and, wherein the location of the thrombus is converted by the MRI-bases host computer into MRI coordinates using a registration transformation.

It should be understood that the foregoing description is merely illustrative of the present disclosure. Various alternatives and modifications can be devised by those skilled in the art without departing from the scope or spirit of the disclosure.

A further method of treating deep vein thrombosis in a peripheral vasculature of a patient, the method comprising: percutaneously accessing a venous vessel of a patient with an introducer sheath through an access site into the venous vessel of the patient; inserting a catheter constraining a thrombectomy device through the lumen of the introducer sheath so that a distal tip of the catheter is distally past a portion of the thrombus ; deploying the thrombectomy device from a constrained configuration to an expanded configuration, wherein the thrombectomy device is in an expanded state between about 20 degrees and about 50 degrees; and, removing the thrombectomy device from the patient.

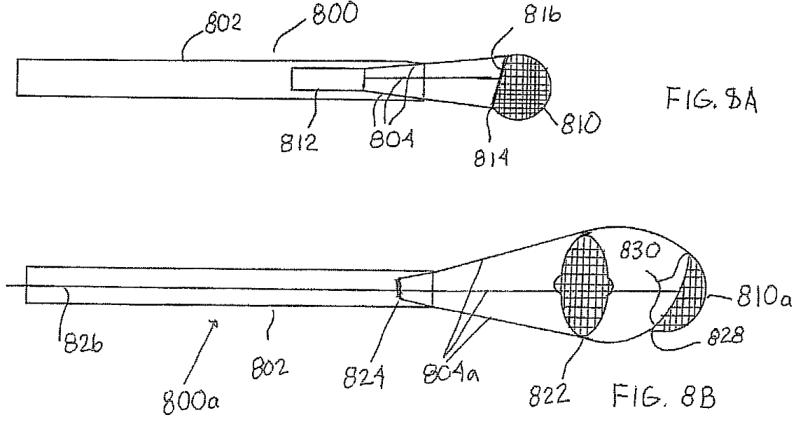

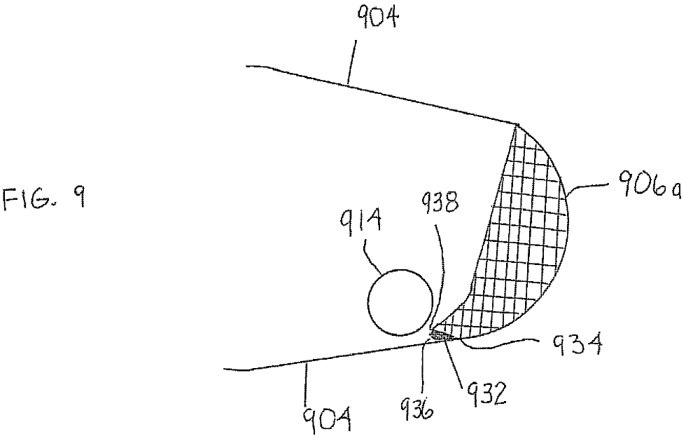

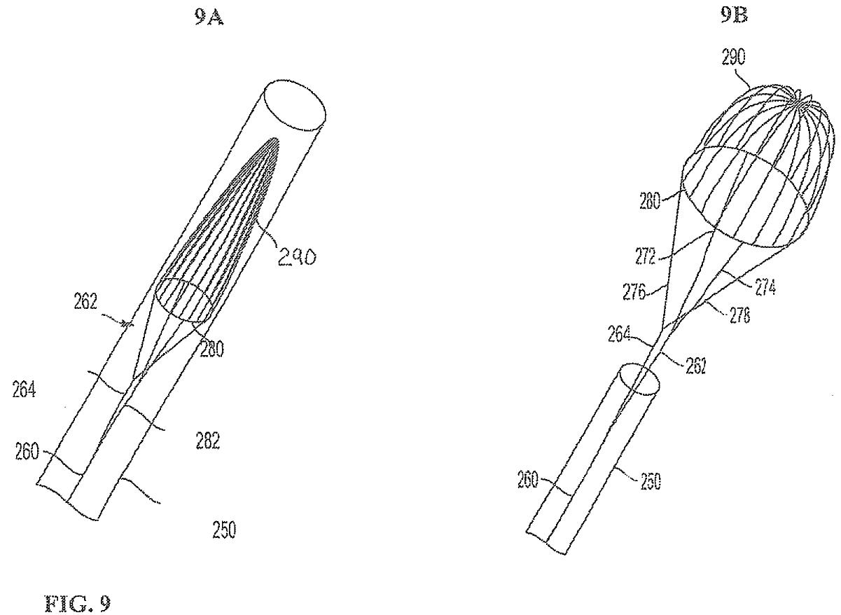

FIG. 8 shows a mesh web wire system 800 having different protruding elements. protrusions, dots, or gripping elements (804, 806, 808, 810, 812, 814, 816 818) that are on the wire 802, generally facing inward (towards where a clot or a thrombus would be in contact with the wire 802. Different structures for these protruding elements are exemplified separately to show how the different shapes and dimensions and configurations can be selected to provide unique and designed functions with respect to different types of clots, thrombus and debris based on size, texture, rheology and dimensions of the unwanted materials to be captured. Protrusion 804 is a generic and simplest protrusion to form and likely the easiest to manufacture, comprising a truncated spherical dot.

Element 806 is a pyramidal element, with a more pointed tip to grasp thrombus with texture and hard surfaces. Element 808 has inwardly sloped side rising to a flat surface to grasp softer clots. Element 810 has inwardly sloped side rising to a concave surface 810a to grasp soft clots and with edges of the concave surface grasping into the clot, yet retaining a large surface area of contact with the particle to be removed.

Element 812 is again a relatively generic and simple protrusion to form. Element 814 is shown with outwardly sloped sides 814a which can be used to trap smaller particles as the wire mesh is withdrawn, the sloped sides capturing particles that might even escape the wire mesh. Element 816 is a truncated spherical element, with the cut through the sphere sufficiently low as to again create inwardly sloped surfaces 816a which may provide the small particle capture function described for element 814 above. Element 818 is shown with a textured surface 818a which can assist in grasping clots that might have smoother or more slippery surfaces.

A textured, grooved, irregular surface such as in 818a can be provided on any of the individual structures. Many techniques for forming such surfaces such as embossing, leaching of soluble materials (e.g., soluble polymers, salts, sugars, etc.) in the deposited metal, ceramic, composite or polymeric elements, and the like.

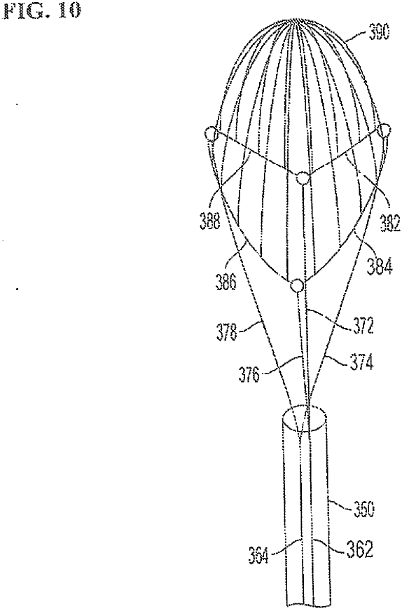

FIG. 9 shows a side view of a shaped distal mesh element 906a capturing a clot 914. Only two struts 904 are shown for convenience. The lower of the two struts shown as 904 is connected to the distal mesh element 906a just beyond a feature 932 on a bottom portion of the distal mesh element 906a. This feature 932 would likely slide along a wall of a blood vessel (not shown), with a curved leading section 936 to minutely elevate the leading section 936 so as to avoiding plowing into or scraping the blood vessel. The extreme front of this leading section 938 may revert backwards to form a lifting function on the thrombus 914. That extreme front of this leading section 938 may be flat or curved (preferably convex curve, but concave may provide an additional function).

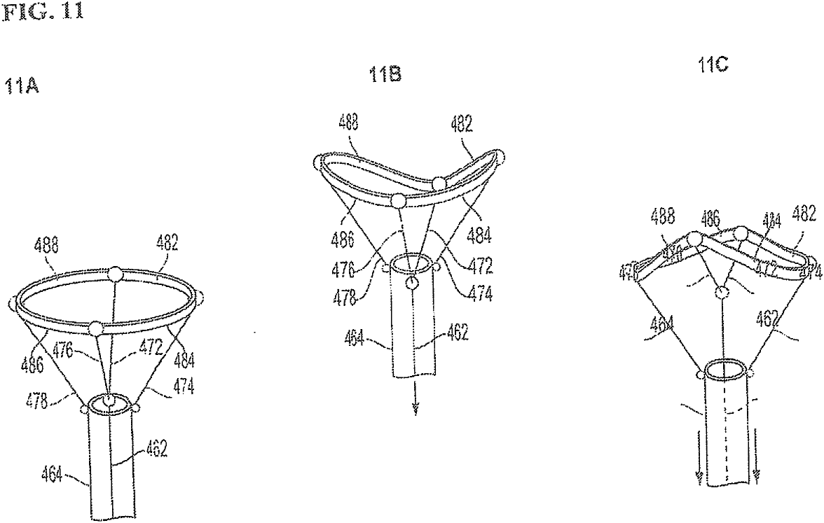

FIG. 10 shows a fully deployed capture system (with the crosswires in the capture web 390 not shown to facilitate other distinguishing elements. The circular ring-shaped element is circular in an aspect view, but from this side view, the connection points for the distal guidewires 372, 374, 376, 378 to the segments of the ring-shaped structure 382, 384, 386, 388 are not within a single plane. The primary retraction guidewires 362, 364 are shown still within the deployment catheter 350.

FIG. 11 shows three different aspects or perspectives of the deployed ring-shaped structure’s four segments 482, 484, 486, 488 secured by four distal guidewires 472, 474, 476, 478 with a single primary guidewire 462 drawing or releasing the ring-shaped structure and progressively allowing the ring-shaped structure to deform or collapse as progressively shown in FIGS. 11A, 11B and 11C.

FIG. 12 shows a different perspective of the deployed ring-shaped structure segments 582, 584, 586, 588 with the connection points 583, 585, 587, 589 connecting the segments to the distal guidewires 572, 574, 576, 578 which are in turn connected to primary retraction guidewires 562, 564 within the catheter 550.

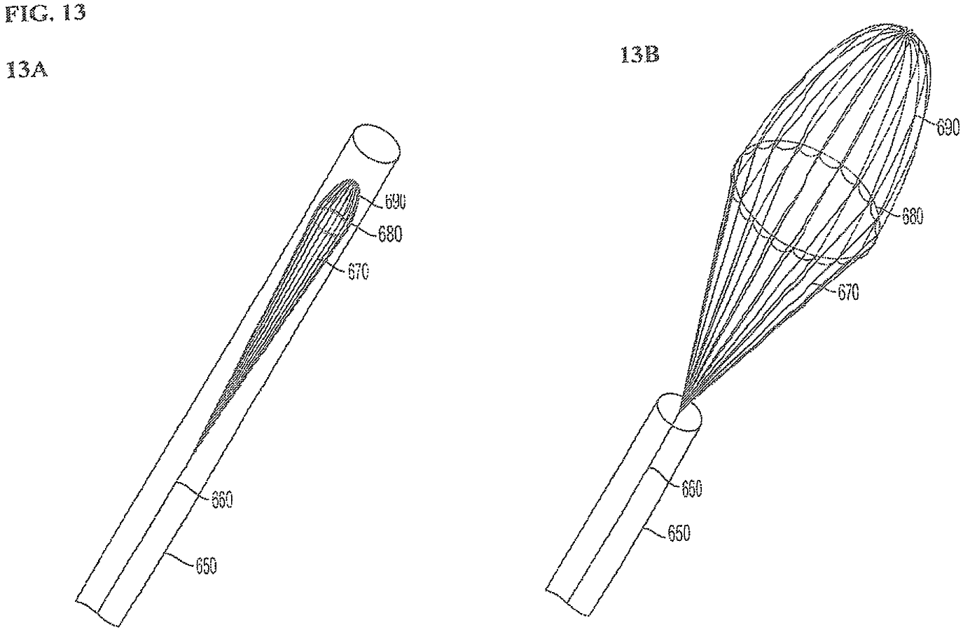

FIG. 13 (13A, 13B) shows a pre-deployed 13A and deployed 13B catheter delivery assembly comprising a catheter 650, single retraction guidewire 660, multiple dual wire element distal retraction guidewires 670, the dual wire element distal retraction guidewires 670 being attached to sides of struts 680 forming the ring-shaped circular opening supporting elements for the wire mesh web 690 (shown without cross wire hatching).

FIG. 14A shows a pre-deployed advanced catheter delivery system 900 with three deployable, expandable elements 906, 908 and 910. There are at least three struts 904 within the deploying catheter 902 which are also carried, along with deployable, expandable elements 906, 908 and 910 within the deployment catheter 900. Clot 914 is shown relative to the various stages of deployment and capture by the device.

FIG. 14B shows a partially deployed advanced catheter delivery system 900 with three deployed, expandable elements 906, 908 and 910.

FIG. 14C shows a fully deployed advanced catheter delivery system 900 with three deployed, expandable elements 906, 908 and 910 still not expanded.

FIG. 14D shows a fully deployed advanced catheter delivery system 900 with three deployed, expandable elements 906, 908 and 910 now expanded as forward (or distal) deployed retracting basket 906a, intermediate expander/containing-cover 908a (for sealing 906a), and the proximal retrieving basket 910. The at least three deployed and expanded struts 904a, 904b and 904c maintain and/or initiated/assisted the expansion of deployed, expanded elements 906a, 908a and 910a into the size and shape necessary to capture clot 914.

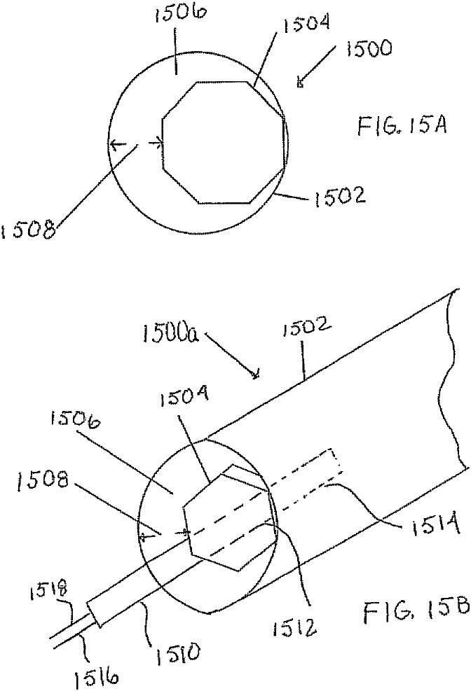

FIG. 15A shows an axial view 1500 of a blood vessel 1502 with a clot 1504 attached more against a single wall. There is a general open area 1506 within the blood vessel 1502, and a greatest distance 1508 between the clot 1504 and the wall 1502. In that area, noninvasive imaging and analytic techniques (not shown) should be used to determine an effective, preferred or best position for insertion of the delivery device 1510 in the greatest distance 1508 between the clot 1504 and the wall 1502. The operating technician or physician will put the delivery catheter in the most appropriate position, between the clots and the blood vessel wall, using the non-invasive visualization as mapping and directions, preferably under live visualization of the region.

FIG. 15B shows a perspective view 1500a of the blood vessel 1502 and clot 1504 of 15A with the deployment device 1510 inserted and positioned past the clot 1504, but no expandable elements (not shown) deployed. The deployment device beneath 1512 and beyond 1514 the clot 1504 is shown. It has passed through the greatest distance 1508 between the clot 1504 and the wall 1502. Additionally, a guide wire 1516 and an conductive wire 1518 are shown. The conductive wire 1518 may be used to operate elements or sub-devices (not shown) or release drugs or heat elements associated with the thrombectomy device of the present invention before or after deployment.

In the following figure and description of the operation of the expander, and additional function is available. The expander may be repeatedly expanded and deflated, forcing a surface of the expander against he clot to compress the clot and to assist in breaking up the clot within the captured environment of the forward collection mesh. The proximal side of the expander (closest to the delivery catheter) may be more rigid and less expandable once initially expanded. This can be effected by having a more dense, higher density mesh, or less flexible composition that the distal side of the expander. The face of the expander facing and engaging the clot may also be textured or have prongs or miniature blades thereon to assist in breaking up the clot.

The distal side of the expander undergoes macrostructural expansion and contraction, no merely thermal expansion which solids undergo. This can be done with motors, pumps, electrical rearrangement of structural elements, inflation-deflation of small balloons, and other known phenomena.

FIG. 15 shows the distal mesh element 1502 and the intermediate mesh element 1508 with a clot 1514 there between. The intermediate mesh element 1508 has an additional expandable element 1520 that when expanded in size to 1522 can impact the clot 1514 within the expanded distal mesh element 1502. The additional expandable element 1520 expands in a direct 1526 against the clot 1514.

FIG. 15 shows a thrombectomy net system 1500 with an expander 1508 that when expanded to 1522, acts to provide at least additional surface pressure which acts to break apart a thrombus 1514 that has been caught in between the distal mesh element collecting basket 1502 and the middle mesh element 1508 (referenced herein as the expander). The collecting basket 1502 and middle mesh expander element 1508 are initially expanded and then partially held open by struts with elastic memory 1504.

When the thrombus 1514 is contained within the holding basket 1502, a signal, such as an electrical signal (or radio frequency signal) is sent through an electrical wire 1524 to the expander 1508. Upon receiving this signaling, a responsive system in the expander 1508, the forward-facing face 1520 of the expander 1508 is extended with more force to provide at least pressure into the thrombus 1504 within the collecting basket 1502, assisting in breaking the thrombus 1514 apart, and allowing for its easier removal, as by retraction of the entire device and clot fragments into the delivery catheter, with or without aspiration of the area by additional device.

In an alternate embodiment, the expander 1508 has a further distinctly expandable element 1520, such as a quasi-balloon structure that expands (e.g., see 1522) upon receiving externally generated signals. The signals may be operated by a technician in the operating environment, as previously noted by electrical, RF or other signaling modality.

For example, the signals may be through wire 1524. In another alternate embodiment, the expander 1508 is a magnetic device that uses electromagnetic forces upon receiving power from wire 1524 that pushes the expanding section 1520 of the expander 1508 into/against the thrombus 1514. This not only secures the thrombus within the collecting basket 1502, but may also act in breaking the clot apart, and assisting in its removal.

As noted, the forward face of the expanding section 1522 (moving in direction 1526) may have protuberances, edges, thin blades, posts, etc. to penetrate into the thrombus, helping to break it into smaller pieces that may be able to be withdrawn into the lumen/cannula of the delivery catheter. There may even be a second signal causing these forward face additions to vibrate, further enhancing clot breakage. This may be done by a small vibrator (not shown) attached to the innards of expanding section 1522 or even within the expander 1508.

In addition to having expanding section 1520, 1522 as a balloon, with a pump or fluid access to expand it on demand, other technologies such as component rigidifying are known in the art.

U.S. Pat. No. 11,344,250 (Mou) describes an expandable electrophysiology catheters having electrodes mounted on splines of an expandable member. The splines of the expandable member include subsegments between a proximal location and a distal intersection at a central axis. The subsegments can include respective top-down profiles, and at least one of the top-down subsegment profiles is straight between the central axis and an adjacent top-down subsegment profile. The subsegments can be interconnected to extend continuously about the central axis from the proximal location to the distal intersection. Other embodiments are also described. This technology may be used to have expanding section 1612 expand on external demand.

Similarly, Piskarev et al. Wiley Online Library in “A Variable Stiffness Magnetic Catheter Made of a Conductive Phase-Change Polymer for Minimally Invasive Surgery,” first Published 06 Feb. 2022 at https://doi.org/10.1002/adfm.202107662, describes variable stiffness (vs) is an important feature that significantly enhances the dexterity of magnetic catheters used in minimally invasive surgeries, existing magnetic catheters with vs consist of sensors, heaters, and tubular structures filled with low melting point alloys, which have a large stiffness change ratio but are toxic to humans, in this paper, a vs magnetic catheter is described for minimally invasive surgery; the catheter is based on a novel variable stiffness thread (vst), which is made of a conductive shape memory polymer (csmp). the csmp is nontoxic and simultaneously serves as a heater, a temperature sensor, and a vs substrate, the vst is made through a new scalable fabrication process, which consists of a dipping technique that enables the fabrication of threads with the desired electrical resistance and thickness (with a step size of 70 µm). selective bending of a multisegmented vst catheter with a diameter of 2.0 mm under an external magnetic field of 20 mt is demonstrated, compared to existing proof-of-concept vs catheters for cardiac ablation, each integrated vst segment has the lowest wall thickness of 0.75 mm and an outer diameter of 2.0 mm. the segment bends up to 51° and exhibits a stiffness change factor of 21. This also may be used for expanding section 1612 expand on external demand.

The method may be practiced wherein before bringing the stabilized blood clot and the most distal mesh element and the middle one of the mesh elements towards the most proximal of the three separate and distinct compressed and expandable mesh elements, a relative distance between the most distal mesh element and the middle one of the mesh elements is repeatedly altered to assist in positioning the stabilized blood clot against the most distal mesh element, and the most distal mesh element is rotated within the blood vessel to position a largest area of the opening which faces towards the interior catheter and the middle one of the three separate and distinct compressed and expandable mesh elements against the stabilized clot. As clots tends to have an asymmetric orientation within the blood vessel, the rotation of the opening of the most distal mesh element is intended to orient and place the forward edge of a sloped opening either against a surface of the clot either closest to the most distal mesh element or farthest from the most distal mesh element. Those orientations can be dependent upon the morphology and rheology of the clots in improving performance. This can be done under live visualization by the surgeon.

Similarly, ACS (American Chemical Society) Publications at https://doi.org/10.1021/acsami.1c06786 evidences another system which may be used for expanding section 1612 expand on external demand, in which Bhuyan et al. illustrate that the Jun. 8, 2021 publication of “Soft and Stretchable Liquid Metal Composites with Shape Memory and Healable Conductivity” in shape memory composites are fascinating materials with the ability to preserve deformed shapes that recover when triggered by certain external stimuli. Although elastomers are not inherently shape memory materials, the inclusion of phase-change materials within the elastomer can impart shape memory properties. When this filler changes the phase from liquid to solid, the effective modulus of the polymer increases significantly, enabling stiffness tuning. Using gallium, a metal with a low melting point (29.8° C.), it is possible to create elastomeric materials with metallic conductivity and shape memory properties. This concept has been used previously in core-shell (gallium-elastomer) fibers and foams, but here, we show that it can also be implemented in elastomeric films containing microchannels. Such microchannels are appealing because it is possible to control the geometry of the filler and create metallically conductive circuits. Stretching the solidified metal fractures the fillers; however, they can heal by body heat to restore conductivity. Such conductive, shape memory sheets with healable conductivity may find applications in stretchable electronics and soft robotics.

In determining treatments and tools to be used, although clot density is important, thrombus characteristics may only be a small part of the story, contingent on time since stroke onset, individual arterial anatomy, and presence of collaterals. We have previously suggested that stasis of thrombus in an intracranial vessel may rapidly produce a secondary thrombus around the original one that is RBC rich, particularly if there are poor collaterals or local angioarchitecture. Consistent with this, slow collateral flow can be associated with thrombus extension in large artery occlusions.^{e95} This may explain the inability to identify a relationship between RBC composition and arteriogenic or cardioembolic stroke etiology, as the new thrombus components may constitute a significant portion of the overall thrombus, with the proportion of RBC in thrombus depending on timing from stroke onset.

The RBC-rich nature of a static, proximal, hyperacute thrombus is correlated with increased hyperdensity on NCCT and blooming artifact on MRI; longer clots may have more hyperdensity and blooming due to freshly formed clot extension within the intracranial vessel. However, we also know there is a time-dependent loss of density in occluded M1 segments within the first few hours. Therefore, depending on timing of patient presentation and imaging, thrombi may have very different composition and imaging characteristics. The lower stiffness and friction and increased permeability of the initial RBC component may facilitate higher penetration of thrombolytic agent and easier extraction. However, over time, more extensive fibrin deposition and crosslinking between RBC and fibrin may occur to facilitate stabilization of thrombus, as evidenced by RBC projections that allow interaction with each other and with fibrin fibers. Inhibitors of tPA may also play more of a role over time. All these factors may work to delay fibrinolysis and increase lytic resistance (a summary of dynamic changes in thrombus composition and imaging findings from time of stroke onset and visualization of these alterations on clot lysability).

The method of the claimed and disclosed invention bay be used in procedures where both hard and soft clot are removed in an acute ischemic stroke patient, deep vein thrombosis patient or pulmonary embolism patient in a single pass without damaging the intima of an occluded blood vessel. That method and other methods disclosed herein may have at least one of a shear-wave dispersion ultrasound and an ultrasonic sensor configured to determine thrombus composition is used to measure ultrasound transducer properties of soft and hard clots before inserting the delivery catheter into the region within the blood vessel without passing the position of the blood. Additionally, the method disclosed herein may have an an artificial intelligence function stored in memory and machine learning components configured to evaluate composition of retrieved soft and hard blood clots uses data derived from the at least one of a shear-wave dispersion ultrasound and an ultrasonic sensor to determine composition of to be retrieved soft and hard blood clots. In the use of the method of claim with the mechanical thrombectomy catheter, it may be combined with an aspiration device controlled by artificial intelligence and machine learning. The use of artificial intelligence and machine learning components can increase first-pass recanalization in a combined mechanical thrombectomy catheter aspiration device. The method may have artificial intelligence and machine learning to assist in visualizing thrombectomy catheters without fluoroscopy that can be superimposed on roadmap views of the vasculature and pathology before, during and after procedures. All of these methods may use sensors that can be connected to a host computer wherein diagnostic algorithms can be used to evaluate treatment plans for clot removal in individual patients. The artificial intelligence computer-based tracking system may be used in patients diagnosed with AIS, DVT or PE clots to monitor the age, composition, size and anatomical location of a thrombus, and/or to track blood clots in the body. For example, an artificial intelligence computer-based system may be used with biplane fluoroscopy to improve the accuracy of cerebral angiography and/or to track blood clots in AIS, DVT and PE patients. The artificial intelligence and a machine learning system may be used with a host computer to actively modify the clot treatment algorithm for individual AIS, DVT and PE patients during procedures in which the clots are removed.

What is claimed:

1. A method of removing a blood clot from a position within a blood vessel comprising:
inserting a delivery catheter into a region within the blood vessel without passing the position of the blood clot, the delivery catheter comprising an exterior catheter, and a deployment catheter as an interior catheter within a lumen of the exterior catheter;
the deployment catheter comprises:
a. the interior catheter with a second lumen, and within the second lumen in linear orientation within the second lumen are three separate and distinct compressed and expandable mesh elements connected by at least two struts passing along a surface of a middle one of the compressed and expandable mesh elements;
b. the at least two struts having elastic memory sufficient to expand themselves and provide expansion forces on the three separate and distinct compressed and expandable mesh elements;
c. a most distal one of the three separate and distinct compressed and expandable mesh elements having an opening which faces towards the interior catheter and the middle one of the three separate and distinct compressed and expandable mesh elements after the most distal one of the compressed and expandable mesh elements is deployed and expands;
d. a most proximal one of the three separate and distinct compressed and expandable mesh elements having an opening which faces away from the interior catheter and towards the middle one of the three separate and distinct compressed and expandable mesh elements after the most proximal one of the compressed and expandable mesh elements is deployed and expands;
the method further comprising extending the deployment catheter past the clot while the exterior catheter does not pass the position of the blood clot;
once a leading end of the deployment catheter has passed the position of the blood clot, deploying only the most distal one of the three separate and distinct compressed and expandable mesh elements past the position of the blood clot, while the middle one of and the most proximal one of the three separate and distinct compressed and expandable mesh elements remain between the clot and the interior catheter;
the at least two struts expanding to expand all of the three separate and distinct compressed and expandable mesh elements;
withdrawing the most distal one of the three separate and distinct compressed and expandable mesh elements towards the blood clot and supporting it on or within the opening which faces towards the interior catheter and the middle one of the three separate and distinct compressed and expandable mesh elements, and bringing the most distal mesh element and the middle one of the mesh elements together, with the blood clot stabilized between the most distal mesh element and the middle mesh element;
bringing the stabilized blood clot and the most distal mesh element and the middle one of the mesh elements towards the most proximal of the three separate and distinct compressed and expandable mesh elements;
at least partially withdrawing the deployment catheter into the exterior catheter; and
withdrawing the delivery catheter from the blood vessel along with the blood clot.

2. The method of claim 1 wherein bringing the stabilized blood clot and the most distal mesh element and the middle one of the mesh elements towards the most proximal of the three separate and distinct compressed and expandable mesh elements;
at least partially withdrawing the deployment catheter into the exterior catheter; and
withdrawing the delivery catheter from the blood vessel along with the blood clot are performed sequentially.

3. The method of claim 1 wherein bringing the stabilized blood clot and the most distal mesh element and the middle one of the mesh elements towards the most proximal of the three separate and distinct compressed and expandable mesh elements;
at least partially withdrawing the deployment catheter into the exterior catheter; and
withdrawing the delivery catheter from the blood vessel along with the blood clot are performed contemporaneously.

4. The method of claim 1 wherein a surface of the middle mesh element conformed against a surface of the most distal mesh element.

5. The method of claim 1 wherein there are at least three struts with elastic memory deployed from the interior catheter.

6. The method of claim 1 wherein the opening in the most distal one of the mesh elements forms a surface that is not orthogonal to surfaces within the blood vessel.

7. The method of claim 1 wherein a distal face of the middle one of and the most proximal one of the three separate and distinct compressed and expandable mesh elements has an additional expandable and deflateable element attached thereto, which is expandable and deflateable by external controls, and after deployment and expansion of the middle one of the expandable mesh elements, the front face of the additional expandable and deflatable element is expanded and deflated to apply force against the clot to assist in reducing total volume of the clot.

8. The method of claim 7 wherein the front face of the additional expandable and deflatable element is repeatedly expanded and deflated.

9. The method of claim 7 wherein the front face of the additional expandable and deflatable element has at least one protuberance, prongs, projection, blade or edge to assist in disrupting the clot.

10. The method of claim 8 wherein a region surrounding the clot within the blood vessel is non-invasively observed to determine a largest space between the clot and an interior wall of the blood vessel, and the leading end of the deployment catheter is passed through the largest space between the clot and the interior wall of the blood vessel until the leading end of the deployment catheter is past the position of the blood clot.

11. The method of claim 9 wherein a region surrounding the clot within the blood vessel is non-invasively observed to determine a largest space between the clot and an interior wall of the blood vessel, and the leading end of the deployment catheter is passed through the largest space between the clot and the interior wall of the blood vessel until the leading end of the deployment catheter is past the position of the blood clot.

12. A device for the removal of a thrombus from a blood vessel having a delivery catheter as an exterior catheter comprising:
a) a deployment catheter within a first lumen of the exterior catheter, the deployment catheter comprising:
i ) an interior catheter with a second lumen, and within the second lumen in linear orientation within the second lumen are three separate and distinct compressed and expandable mesh elements connected by at least two struts passing along a surface of a middle one of the compressed and expandable mesh elements;
ii) the at least two struts having elastic memory sufficient to expand themselves and provide expansion forces on the three separate and distinct compressed and expandable mesh elements;
iii) a most distal one of the three separate and distinct compressed and expandable mesh elements having an opening which faces towards the interior catheter and the middle one of the three separate and distinct compressed and expandable mesh elements after the most distal one of the compressed and expandable mesh elements is deployed and expands; and
iv) a most proximal one of the three separate and distinct compressed and expandable mesh elements having an opening which faces away from the interior catheter and towards the middle one of the three separate and distinct compressed and expandable mesh elements after the most proximal one of the compressed and expandable mesh elements is deployed and expands.

13. The device of claim 12 wherein there are at least three and fewer than six struts having elastic memory sufficient to expand themselves and provide expansion forces on the three separate and distinct compressed and expandable mesh elements.

14. The device of claim 12 wherein the middle one of the three separate and distinct compressed and expandable mesh elements has mesh surfaces in both a proximal face and a distal face when expanded.

15. The method of claim 1 wherein before bringing the stabilized blood clot and the most distal mesh element and the middle one of the mesh elements towards the most proximal of the three separate and distinct compressed and expandable mesh elements, a relative distance between the most distal mesh element and the middle one of the mesh elements is repeatedly altered to assist in positioning the stabilized blood clot against the most distal mesh element, and the most distal mesh element is rotated within the blood vessel to position a largest area of the opening which faces towards the interior catheter and the middle one of the three separate and distinct compressed and expandable mesh elements against the stabilized clot.

16. The method of claim 15 wherein the rotation of the most distal mesh element and securing of the stabilized clot between the most distal mesh element and the middle one of the three separate and distinct mesh elements is done under live visualization.

17. The method of claim 1 wherein both hard and soft clot are removed in an acute ischemic stroke patient, deep vein thrombosis patient or pulmonary embolism patient in a single pass without damaging the intima of an occluded blood vessel.

18. The method of claim 1 wherein at least one of a shear-wave dispersion ultrasound and an ultrasonic sensor configured to determine thrombus composition is used to measure ultrasound transducer properties of soft and hard clots before inserting the delivery catheter into the region within the blood vessel without passing the position of the blood.

19. The method of claim 18 wherein an artificial intelligence stored in memory and machine learning components configured to evaluate composition of retrieved soft and hard blood clots uses data derived from the at least one of a shear-wave dispersion ultrasound and an ultrasonic sensor to determine composition of to be retrieved soft and hard blood clots.

20. The method of claim wherein 19 the mechanical thrombectomy catheter combined with an aspiration device controlled by artificial intelligence and machine learning.
